(19) United States
(12) Patent Application Publication
(10) Pub. No.: US 2014/0147890 A1
(43) Pub. Date: May 29, 2014

(54) GENETICALLY MODIFIED PHAGE AND USE THEREOF

(71) Applicant: Cedric Szpirer, Fleurus (BE)

(72) Inventor: Cedric Szpirer, Fleurus (BE)

(73) Assignee: DELPHI GENETICS, Gosselies (BE)

(21) Appl. No.: 14/131,320

(22) PCT Filed: Jul. 6, 2012
(86) PCT No.: PCT/EP2012/063244

§ 371 Date: Jan. 7, 2014

Related U.S. Application Data
(63) Continuation-in-part of application No. 13/178,402, filed on Jul. 7, 2011.

(30) Foreign Application Priority Data
Jul. 7, 2011 (EP) 11173033.9

(51) Int. Cl. C12N 15/73

(52) U.S. Cl. CPC C12N 15/73
USPC 435/69.1; 435/236; 435/252.33; 435/472

(57) ABSTRACT

The present invention relates to a genetically modified phage and use thereof in a method for producing a bio molecule of interest.

### FIELD OF INVENTION

The present invention relates to the field of production of biomolecules of interest in biologic systems. Specifically, the present invention relates to a genetically modified phage, and use thereof in order to avoid phage contamination of the culture broth and/or bacterial lysis.

### BACKGROUND OF INVENTION

Bacterial cells are systems of choice for the production of biomolecules, especially of target proteins. Among other advantages, bacterial systems are easy to use and allow the rapid production of large quantities of proteins in a limited volume of culture.

One of the most widely and routinely used bacterial system is the bacteriophage T7 expression system. This bacterial system was described in U.S. Pat. No. 4,952,496. In this system, the gene encoding the target protein is placed under the control of a T7 promoter, and is transformed in a bacterial host, usually E. coli, which comprises an integrated lambda DE3 lysogen phage. The lambda DE3 lysogen phage carries the gene encoding the T7 RNA polymerase under the control of a lacUV5 promoter. When cultured on an IPTG-containing medium, the expression of the T7 RNA polymerase is induced, and allows the expression of the target protein.

Due to the integration of the gene of the T7 RNA polymerase (T7 gene 1) within the sequence of the Int gene, Lambda DE3 phage should be defective in its ability to enter into the lytic phase. However, bacterial lysis is observed during some protein productions and in the absence of any other phage, suggesting that the DE3 phage may recover its lytic properties. The bacterial lysis and even more, the presence of infectious phages in the culture broth is highly problematic because (i) it compromises the use of produced target proteins for some applications, such as, for example, pharmaceutical applications, (ii) the decontamination process in order to remove any trace of phages requires the shutdown of the production lines and the complete renewal of the batches of culture and (iii) it reduces dramatically the yield of recombinant protein.

Alternative methods to the use of a phage have been described:

WO03/050240 describes an expression system for producing a target protein in a host cell comprising a gene encoding T7 RNA polymerase integrated using homologous recombination. However, the system of WO03/050240 is difficult to implement, due to the size of the T7 RNA polymerase gene and due to the fact that homologous recombination is not easy to do in all E. coli strains (as mentioned by Phue et al., Biotechnology and Bioengineering, 101, 831-836, 2008). Consequently, the number of transformed cells carrying the T7 RNA polymerase integration remains very low or these cells are not obtained. Moreover, using homologous recombination, it is necessary to use a selective marker to select bacteria containing integration of the T7 RNA polymerase gene. This marker will not be usable for another selection step and could be undesired for the final use of the strain. For example, selective markers often used are antibiotic resistance genes but it is recommended to avoid these genes in biopharmaceutical productions. An additional step is thus required to remove the antibiotic resistance gene from the strain and it is not always possible to do it.

WO2008/139153 describes another expression system, wherein the host cell is transformed with a plasmid comprising an expression cassette for T7 RNA polymerase. However, due to the use of a plasmid, hosts cells have to be maintained in selective conditions to make sure that they still comprise the plasmid. In addition, plasmids are frequently subjected to recombination, which impaired the expression system.

Therefore, there is a need for a novel method for producing a biomolecule of interest, wherein, when a phage is used, the culture is not contaminated by infectious phages or unintentionally lysed during growth or protein production.

The present invention hereby provides a genetically modified phage that does not recover its lytic properties during culture, thereby allowing the production of a biomolecule of interest without phage contamination.

### SUMMARY

One object of the invention is a genetically modified phage wherein
an expression system is inserted,
the S and/or the R genes are inactivated.

In one embodiment of the invention, the Int and/or Xis gene is inactivated.

Another object of the invention is a genetically modified phage wherein
an expression system is inserted,
the S, R and/or the Q genes are inactivated, and
the Int and/or Xis gene is inactivated.

In another embodiment of the invention, the Rz gene is inactivated.

In another embodiment of the invention, an expression system is inserted and the Int gene, the Xis gene and the R, S and Rz genes are inactivated.

In another embodiment of the invention, an expression system is inserted and the Int gene, the Xis gene and the Q, R, S and Rz genes are inactivated.

In another embodiment of the invention, the expression system is the T7 expression system.

In another embodiment of the invention, the phage is the lambda phage, the 434phage, the phi80 phage, the phi81 phage, the HK97 phage, the P21 phage.

In another embodiment of the invention, the genetically modified phage as described here above has the sequence SEQ ID NO: 10. In another embodiment of the invention, the genetically modified phage as described here above is one of the phages P11 to P53.

Another object of the invention is a kit comprising the genetically modified phage as described here above and a helper phage.

Another object if the invention is a host cell comprising the genetically modified phage as described here above.

In one embodiment of the invention, the host cell is an enterobacteria, preferably E. Coli, more preferably BL21.

In another embodiment of the invention, the host cell as described here above further comprises the inactivation of at least one of the genes tonA, galK, araB, araA, lon, ompT, rcsA, hsdR, mrr, endA and recA.

In another embodiment of the invention, the host cell as described here above comprises the insertion of the ccdb gene.

Another object of the invention is a kit comprising the host cell as described here above and a plasmid comprising the ccdA gene.

Another object of the invention is a process for preparing a host cell as described here above, comprising infecting a host cell with a genetically modified phage as described here above.

Another object of the invention is a process for producing a biomolecule of interest, comprising
cultivating a host cell comprising the genetically modified phage according to any one of claims 1 to 7 and the nucleic acid sequence of the biomolecule of interest,
recovering the biomolecule of interest.

### DETAILED DESCRIPTION

The present invention relates to a genetically modified phage, wherein the ability of the phage to regain its lytic properties is limited.

The Inventors focused on the genetic modification of a phage. Surprisingly, the Inventors showed that it was not possible to delete all viral sequences of the integrated phage, because the viability of the infected bacteria was severely compromised in that case. In particular, the Inventors showed that the deletion of a DNA fragment comprising the coding sequences of the ral gene and the N gene leads to the death of the host cell (See EXAMPLES). This result was surprising because the ral and N genes are not known to be involved in the lysogenic state; the N gene is only described as essential for lytic growth. On the contrary, in lysogenic state, a repressor of the phage, named CI or C2, blocks the expression of N and ral.

The present invention thus relates to a genetically modified phage wherein
an expression system is inserted, and
at least one of the Int, Xis, R, S, Q and Rz genes is inactivated.

In one embodiment, the genetically modified phage comprises an expression system and the S gene is inactivated.

In one embodiment, the genetically modified phage comprises an expression system and the R gene is inactivated.

In one embodiment, the genetically modified phage comprises an expression system and the Q gene is inactivated.

In one embodiment, the genetically modified phage comprises an expression system and the Int gene is inactivated.

In one embodiment, the genetically modified phage comprises an expression system and the Xis gene is inactivated.

In one embodiment, the genetically modified phage comprises an expression system and the S and Int genes are inactivated.

In one embodiment, the genetically modified phage comprises an expression system and the S and Xis genes are inactivated.

In one embodiment, the genetically modified phage comprises an expression system and the S and R genes are inactivated.

In one embodiment, the genetically modified phage comprises an expression system and the S and Rz genes are inactivated.

In one embodiment, the genetically modified phage comprises an expression system and the S and Q genes are inactivated.

In one embodiment, the genetically modified phage comprises an expression system and the R and Rz genes are inactivated.

In one embodiment, the genetically modified phage comprises an expression system and the R and Int genes are inactivated.

In one embodiment, the genetically modified phage comprises an expression system and the R and Xis genes are inactivated.

In one embodiment, the genetically modified phage comprises an expression system and the R and Q genes are inactivated.

In one embodiment, the genetically modified phage comprises an expression system and the Q and Rz genes are inactivated.

In one embodiment, the genetically modified phage comprises an expression system and the Q and Int genes are inactivated.

In one embodiment, the genetically modified phage comprises an expression system and the Q and Xis genes are inactivated.

In one embodiment, the genetically modified phage comprises an expression system and the S, Int and Xis genes are inactivated.

In one embodiment, the genetically modified phage comprises an expression system and the S, Int and Rz genes are inactivated.

In one embodiment, the genetically modified phage comprises an expression system and the S, Int and R genes are inactivated.

In one embodiment, the genetically modified phage comprises an expression system and the S, Xis and Rz genes are inactivated.

In one embodiment, the genetically modified phage comprises an expression system and the S, Xis and R genes are inactivated.

In one embodiment, the genetically modified phage comprises an expression system and the S, R and Rz genes are inactivated.

In one embodiment, the genetically modified phage comprises an expression system and the S, R and Q genes are inactivated.

In one embodiment, the genetically modified phage comprises an expression system and the S, Q and Rz genes are inactivated.

In one embodiment, the genetically modified phage comprises an expression system and the S, Q and Int genes are inactivated.

In one embodiment, the genetically modified phage comprises an expression system and the S, Q and Xis genes are inactivated.

In one embodiment, the genetically modified phage comprises an expression system and the R, Int and Xis genes are inactivated.

In one embodiment, the genetically modified phage comprises an expression system and the R, Int and Rz genes are inactivated.

In one embodiment, the genetically modified phage comprises an expression system and the R, Xis and Rz genes are inactivated.

In one embodiment, the genetically modified phage comprises an expression system and the R, Q and Rz genes are inactivated.

In one embodiment, the genetically modified phage comprises an expression system and the R, Xis and Q genes are inactivated.

In one embodiment, the genetically modified phage comprises an expression system and the R, Int and Q genes are inactivated.

In one embodiment, the genetically modified phage comprises an expression system and the Q, Int and Rz genes are inactivated.

In one embodiment, the genetically modified phage comprises an expression system and the Q, Int and Xis genes are inactivated.

In one embodiment, the genetically modified phage comprises an expression system and the Q, Xis and Rz genes are inactivated.

In one embodiment, the genetically modified phage comprises an expression system and the S, Int, Xis and Rz genes are inactivated.

In one embodiment, the genetically modified phage comprises an expression system and the S, Int, Xis and R genes are inactivated.

In one embodiment, the genetically modified phage comprises an expression system and the S, Int, R and Rz genes are inactivated.

In one embodiment, the genetically modified phage comprises an expression system and the S, R, Xis and Rz genes are inactivated.

In one embodiment, the genetically modified phage comprises an expression system and the R, Rz, Xis and Int genes are inactivated.

In one embodiment, the genetically modified phage comprises an expression system and the Q, R, Rz and S genes are inactivated.

In one embodiment, the genetically modified phage comprises an expression system and the Q, R, Rz and Int genes are inactivated.

In one embodiment, the genetically modified phage comprises an expression system and the Q, R, S and Int genes are inactivated.

In one embodiment, the genetically modified phage comprises an expression system and the Q, Rz, S and Int genes are inactivated.

In one embodiment, the genetically modified phage comprises an expression system and the Q, R, Rz and Xis genes are inactivated.

In one embodiment, the genetically modified phage comprises an expression system and the Q, R, S and Xis genes are inactivated.

In one embodiment, the genetically modified phage comprises an expression system and the Q, Rz, S and Xis genes are inactivated.

In one embodiment, the genetically modified phage comprises an expression system and the Q, R, Int and Xis genes are inactivated.

In one embodiment, the genetically modified phage comprises an expression system and the Q, Rz, Int and Xis genes are inactivated.

In one embodiment, the genetically modified phage comprises an expression system and the Q, S, Int and Xis genes are inactivated.

In one embodiment, the genetically modified phage comprises an expression system and the S, R, Rz, Xis and Int genes are inactivated.

In one embodiment, the genetically modified phage comprises an expression system and the S, R, Q, Rz and Xis genes are inactivated.

In one embodiment, the genetically modified phage comprises an expression system and the S, R, Q, Rz and Int genes are inactivated.

In one embodiment, the genetically modified phage comprises an expression system and the S, R, Q, Xis and Int genes are inactivated.

In one embodiment, the genetically modified phage comprises an expression system and the S, Q, Rz, Xis and Int genes are inactivated.

In one embodiment, the genetically modified phage comprises an expression system and the R, Q, Rz, Xis and Int genes are inactivated.

In one embodiment, the genetically modified phage comprises an expression system and the S, R, Q, Rz, Xis and Int genes are inactivated.

Examples of phages which can be used in the invention include, but are not limited to, the lambda (λ) phage, lambda-like and lambdoid phages. Lambda phage, also known as coliphage lambda, is a virus that infects Escherichia coli. Lambda is a temperate bacteriophage. Lambda-like phages form a family of bacteriophages and archaeal viruses which are characterized by long, non-contractile tails. Lambdoid phages are natural relatives of lambda phage. Most of them grow on E. coli, but a few come from other host cells, such as, for example, Salmonella typhimurium. These phages may have the same gene order as lambda.

Examples of lambda-like and lambdoid phages which could be used in the present invention include, but are not limited to, coliphage 434, phi80, phi81, HK97, P21 and P22.

In an embodiment, the phage is lambda (Enterobacteria phage lambda, accession number NC_{—}001416). The organization of the genome of the lambda phage is shown in table 1 below.

TABLE 1

| Start | End | Name | Description |
|---|---|---|---|
| 191 | 736 | nu1 | DNA packaging protein |
| 711 | 2636 | A | DNA packaging protein |
| 2633 | 2839 | W | head-tail joining protein |
| 2836 | 4437 | B | capsid component |
| 4418 | 5737 | C | capsid component |
| 5132 | 5737 | nu3 | capsid assembly protein |
| 5747 | 6079 | D | head-DNA stabilization protein |
| 6135 | 7160 | E | capsid component |
| 7202 | 7600 | Fi | DNA packaging protein |
| 7612 | 7965 | Fii | head-tail joining protein |
| 7977 | 8555 | Z | tail component |
| 8552 | 8947 | U | tail component |
| 8955 | 9695 | V | tail component |
| 9711 | 10133 | G | tail component |
| 10115 | 10549 | T | tail component |
| 10542 | 13103 | H | tail component |
| 13100 | 13429 | M | tail component |
| 13429 | 14127 | L | tail component |
| 14276 | 14875 | K | tail component |
| 14773 | 15444 | I | tail component |
| 15505 | 18903 | J | tail:host specificity protein |
| 18965 | 19585 | lom | outer host membrane |
| 19650 | 20855 | orf-401 | Tail fiber protein |
| 20767 | 20147 | orf206b | hypothetical protein |
| 21029 | 21973 | orf-314 | Tail fiber |
| 21973 | 22557 | orf-194 | Putative fiber assembly protein |
| 23918 | 22686 | ea47 | ea47 |
| 25399 | 24509 | ea31 | ea31 |
| 26973 | 25396 | ea59 | ea59 |
| 28882 | 27812 | int | integration protein |
| 29078 | 28860 | xis | Excisionase |
| 29285 | 29118 | hypothetical | hypothetical protein |
| 29655 | 29374 | ea8.5 | ea8.5 |
| 30395 | 39847 | ea22 | ea22 |
| 31024 | 30839 | orf61 | hypothetical protein |
| 31196 | 31005 | orf63 | hypothetical protein |
| 31351 | 31169 | orf60a | hypothetical protein |
| 32028 | 31348 | exo | exonuclease |
| 32810 | 32025 | bet | bet |
| 33232 | 32816 | gam | host-nuclease inhibitor protein Gam |
| 33330 | 33187 | kil | host-killing protein |
| 33463 | 33299 | cIII | antitermination protein |
| 35582 | 33494 | ea10 | Putative single-stranded DNA binding |
| | | | protein |
| 35582 | 33930 | ral | restriction alleviation protein |
| 34357 | 34271 | orf28 | hypothetical protein |
| 34482 | 35036 | lambdap48 | Superinfection exclusion protein B |
| 35582 | 34560 | N | early gene regulator |
| 36259 | 35825 | rexb | exclusion protein |
| 37114 | 36275 | rexa | exclusion protein |
| 37940 | 37227 | cI | repressor |
| 38023 | 38135 | cro | antirepressor |
| 38360 | 38653 | cII | transcriptional activator |
| 38686 | 39585 | O | DNA replication protein |
| 39582 | 40283 | P | DNA replication protein |
| 40280 | 40570 | ren | ren exclusion protein |
| 40644 | 41084 | NinB | NinB |
| 41081 | 41953 | NinC | NinC protein |
| 41950 | 42123 | NinD | NinD protein |
| 42090 | 42272 | NinE | NinE protein |
| 42269 | 42439 | NinF | NinF protein |
| 42429 | 43043 | NinG | NinG protein |
| 43040 | 43246 | NinH | NinH protein |
| 43224 | 43889 | NinI | NinI protein |
| 43886 | 44509 | Q | late gene regulator |
| 44621 | 44815 | orf-64 | hypothetical protein |
| 45186 | 45509 | S | Cell lysis protein |
| 45493 | 45969 | R | endolysin |
| 45966 | 46427 | Rz | cell lysis protein |
| 46186 | 46368 | Rz1 | Rz1 protein |
| 46752 | 46459 | bor | Bor protein precursor |
| 47575 | 47042 | lambdap78 | putative enveloppe protein |
| 47738 | 47944 | lambdap79 | hypothetical protein |

In the present invention, the position of the residues within the sequence of the lambda phage relates to NC_{—}001416.

In another embodiment, the phage is lambda DE3 (accession number EU078592). The Lambda DE3 phage is a modified lambda phage D69, comprising the gene encoding the T7 RNA polymerase under the control of a lacUV5 promoter. The list of the genes carried by the sequence of Lambda DE3 and their position are shown in the Table 2 below.

TABLE 2

| Start | End | Name | Description |
|---|---|---|---|
| 341 | 1423 | lacI | lactose operon repressor |
| 1546 | 1995 | lacZ | N-terminal fragment of |
| | | | beta-galactosidase |
| 2026 | 4677 | 1 | T7 DNA-directed RNA polymerase |
| 5804 | 5586 | xis | excisionase |
| 6011 | 5844 | hypothetical | Hypothetical protein |
| 6381 | 6100 | ea8.5 | ea8.5 |
| 7121 | 6573 | ea22 | ea22 |
| 7750 | 7565 | hypothetical | Hypothetical protein |
| 7922 | 7731 | hypothetical | Hypothetical protein |
| 8077 | 7895 | hypothetical | Hypothetical protein |
| 8754 | 8074 | exo | exonuclease |
| 9536 | 8751 | bet | Bet |
| 9958 | 9542 | gam | host-nuclease inhibitor protein Gam |
| 10056 | 9913 | kil | host-killing protein |
| 10189 | 10025 | cIII | antitermination protein |
| 10630 | 10262 | ea10 | putative single-stranded DNA binding |
| | | | protein |
| 11013 | 10813 | ral | restriction alleviation protein |
| 11083 | 10997 | hypothetical | Hypothetical protein |
| 11391 | 11092 | N | probable regulatory protein N (early |
| | | | gene regulator) |
| 12356 | 11706 | C2 | repressor protein C2 |
| 12437 | 12622 | cro | regulatory protein cro (Antirepressor) |
| 12738 | 13037 | cII | antitermination protein |
| 13070 | 13969 | O | DNA replication protein |
| 13966 | 14667 | P | DNA replication protein |
| 14664 | 15467 | ren | Ren exclusion protein |
| 15464 | 16087 | Q | late gene regulator |
| 16199 | 16393 | hypothetical | Hypothetical protein |
| 16764 | 17087 | S | cell lysis protein |
| 17071 | 17547 | R | cell lysis protein |
| 17544 | 18005 | Rz | cell lysis protein |
| 18330 | 18037 | Bor | Bor protein precursor |
| 19153 | 18620 | putative | putative envelope protein |
| 19316 | 19522 | hypothetical | Hypothetical protein |
| 20270 | 20815 | nu1 | DNA packaging protein |
| 20790 | 22715 | A | DNA packaging protein |
| 22712 | 22918 | W | head-tail joining protein |
| 22915 | 24516 | B | capsid component |
| 24497 | 25816 | C | capsid component |
| 25826 | 26158 | D | head-DNA stabilization protein |
| 26214 | 27239 | E | capsid component |
| 27281 | 27679 | Fi | DNA packaging protein |
| 27691 | 28044 | Fii | head-tail joining protein |
| 28056 | 28634 | Z | tail component |
| 28631 | 29026 | U | tail component |
| 29034 | 29774 | V | tail component |
| 29790 | 30212 | G | tail component |
| 30194 | 30628 | T | tail component |
| 30621 | 33182 | H | tail component |
| 33179 | 33508 | M | tail component |
| 33508 | 34206 | L | tail component |
| 34356 | 34955 | K | tail component |
| 34853 | 35524 | I | tail component |
| 35585 | 38983 | J | tail:host specificity protein |
| 39045 | 39665 | lom | outer host membrane |
| 39730 | 40935 | tail | tail fiber protein |
| 41109 | 41372 | tail | tail fiber |
| 42175 | 41237 | ea59 | ea59 |

In the present invention, the position of the residues within the sequence of the lambda DE3 phage relates to EU078592.

As used herein, an “expression system” refers to a linear or a circular DNA molecule composed of a fragment encoding a nucleic acid sequence operably linked to an additional fragment for the transcription of the system.

The additional fragment includes a promoter and a stop codon sequence. The expression system may further contain one or more origins of replication, one or more selection markers and a sequence encoding a ribosome binding site.

“Operably linked” means that fragments are arranged to be functioning as they are supposed to be, for example once transcription starts at the promoter, it goes through coded fragment to stop codon. “Promoter” in the meaning of the present invention is an expression control element that permits binding of RNA polymerase and the initiation of transcription.

In one embodiment of the invention, the nucleic acid sequence is under the control of a “strong” promoter. A strong promoter is characterized by a high binding affinity of the promoter sequence to an RNA polymerase, usually the naturally occurring corresponding RNA polymerase, on the one hand and the rate of formation of mRNA by that RNA polymerase on the other hand.

In a preferred embodiment, the nucleic acid sequence is under the control of an “inducible promoter”. An “inducible promoter” is a promoter that may be regulated by external factors, e.g. the presence of an inductor (also termed “inducer”) molecule or the absence of a repressor molecule, or physical factors like increased or decreased temperature, osmolarity, or pH value. Different promoters and the respective induction principles were reviewed by Makrides et al. (Microbiological Reviews, 1996, (60)3: 512-538). Examples of inducible promoters which may be used in the present invention include, but are not limited to the tac or the trc promoter, the lac or the lacUV5 promoter (all inducible by lactose or its analog IPTG (isopropylthiol-β-D-galactoside)), the tightly regulatable araBAD promoter (PBAD; Guzman et al., 1995, inducible by arabinose), the trp promoter (inducible by β-indole acrylic acid addition or tryptophan starvation, repressible by tryptophan addition), the lambda promoter pL (λ) (induction by an increase of temperature), the phoA promoter (inducible by phosphate starvation), the PprpB (induction with propionate) or other promoters suitable for recombinant protein expression, which all use E. coli RNA polymerase.

Among inducible promoters are those that show a “leaky” expression behavior. Such promoters (so-called “leaky promoters”) are, in principle, inducible, but show nevertheless also basal expression without being externally induced. Inducible promoters that show leaky expression under non-induced conditions may behave similarly to constitutive promoters (i.e. they are steadily and continuously active or they may be activated or enhanced as a result of certain cultivation conditions). Leaky promoters may be particularly useful for continuously operated cultivation processes. Examples of leaky promoters are the T7 promoter and the trp promoter.

In one embodiment of the invention, the promoter may also be constitutive, i.e. a promoter which controls expression without the need for induction on the one hand, or the possibility of repression on the other hand. Hence, there is continuous and steady expression at a certain level. As an example, the strong constitutive HCD promoter (Poo et al., Biotechnology Letters, 2002, 24:1185-1189; Jeong et al., Protein expression and purification, 2004, 36:150-156) may be applied for constitutive expression.

In one embodiment, the expression system comprises a nucleic acid sequence encoding a protein that induces the expression of the biomolecule of interest. Advantageously, the expression of the biomolecule of interest is induced in particular conditions, such as, for example, under selection.

Examples of such nucleic acid sequences include, but are not limited to, the gene encoding the T7 RNA polymerase, T7 gene 1. In that case, the expression of the T7 RNA polymerase induces the expression of the biomolecule of interest placed under the control of a T7 promoter.

Preferably, the expression system is the T7 expression system. The T7 expression system was described in U.S. Pat. No. 4,952,496, which is incorporated herein by reference. The T7 expression system comprises a DNA fragment from the T7 phage, containing the entire coding sequence for the T7 RNA polymerase (i.e. the T7 gene 1). Any natural active promoter of the T7 gene 1 was removed and an inducible lacUV5 promoter was inserted ahead of the coding sequence. The lacUV5 promoter is induced by addition of IPTG to the culture medium.

According to another embodiment, the expression system comprises the nucleic acid sequence of the biomolecule of interest.

With regard to the biomolecule of interest, there are no limitations. It may, in principal, be any amino acid sequences, nucleic acid sequences, such as, for example, DNA or RNA.

Examples of amino acid sequences include polypeptide, protein or peptide that is to be produced on a manufacturing scale, e.g. an industrial biomolecule or a therapeutic biomolecule.

Examples for biomolecules that can be produced by the method of the invention are, without limitation, enzymes, regulatory proteins, receptors, peptides, e.g. peptide hormones, cytokines, membrane or transport proteins.

The biomolecules of interest may also be antigens as used for vaccination, vaccines, antigen-binding proteins, immune stimulatory proteins, allergens, full-length antibodies or antibody fragments or derivatives. Antibody derivatives may be selected from the group of single chain antibodies, (scFv), Fab fragments, Fv fragments, single domain antibodies (VH or VL fragment), domain antibodies like camelid single domain antibodies (VHH, nanobodies) or other antibody formats as described for instance in Andersen and Reilly (Current Opinion in Biotechnology, 2004, 15:456-462) or Holliger and Hudson (Nature Biotechnology, 2005 (23)9: 1126-1136).

The biomolecules of interest in the present invention can also be exemplified by protein (viral antigen), e.g., coat protein, core protein, protease, reverse transcriptase, integrase, and so forth, encoded in the genome of a pathogenic virus, e.g., hepatitis B virus, hepatitis C virus, I-HV, influenza, and so forth; growth factors such as platelet-derived growth factor (PDGF), stem cell growth factor (SCF), hepatocyte growth factor (HGF), transforming growth factor (TGF), nerve growth factor (NGF), epidermal growth factor (EGF), fibroblast growth factor (FGF), insulin-like growth factor (IGF), and so forth; cytokines such as tumor necrosis factor, interferon, interleukin, and so forth; hematopoietic factors such as erythropoietin, granulocyte colony-stimulating factor, granulocyte-macrophage colony-stimulating factor, macrophage colony-stimulating factor, thrombopoietin, and so forth; peptide hormones such as luteinizing hormone-releasing hormone (LB-RH), thyrotropin-releasing hormone (TRH), insulin, somatostatin, growth hormone, prolactin, adrenocorticotropic hormone (ACTH), melanocyte-stimulating hormone (MSH), thyroidstimulating hormone (TSH), luteinizing hormone (LU), follicle-stimulating hormone (FSH), vasopressin, oxytoxin, calcitonin, parathyroid hormone (PTH), glucagon, gastrin, secretin, pancreozymin, cholecystokinin, angiotensin, human placenta lacto8en, human chorionic gonadotropin (HCG), cerulein, motilin, and so forth; analgesic peptides such as enkephalin, endorphin, dynorphin, kyotorphin, and so forth; enzymes such as superoxide dismutase (SOD), urokinase, tissue plasminogen activator (TPA), asparaginase, kallikrein, and so forth; peptide neurotransmitters such as bombesin, neutrotensin, bradykinin, substance P, Alzheimer's amyloid peptide (AD), SOD1, presenillin 1 and 2, renin, Dsynuclein, amyloid A, amyloid P, activin, anti-HER-2, bombesin, enkephalinase, protease inhibitors, therapeutic enzymes, D 1-antitrypsin, mammalian trypsin inhibitor, mammalian pancreatic trypsin inhibitor, calcitonin, cardiac hypertrophy factor, cardiotrophins (such as cardiotrophin-1), CD proteins (such as CD-3, CD-4, CD-8 and CD-19), CFTR, CTNF, DNase, human chorionic gonadotropin, mouse gonadotropin-associated peptide, cytokines, transthyretin, amylin, lipoproteins, lymphokines, lysozyme, a growth hormone (including human growth hormone), bovine growth hormone, growth hormone releasing factor, parathyroid hormone, thyroid stimulating hormone, growth factors, brain-derived neurotrophic growth factor, epidermal growth factor (EGF), fibroblast growth factor (such as D FGF and D FGF), insulin-like growth factor-I and —II, des(1-3)-IGF-I (brain IGF-I), insulin-like growth factor binding proteins, nerve growth factor (such as NGF-D), platelet derived growth factor (PDGF), vascular endothelial growth factor (VEGF), receptors for growth hormones or growth factors, transforming growth factor (TGF) (such as TGF-D, TGF-D 1, TGF-D2, TGF-D3, TGF-D4 or TGF-D5), neurotrophic factors (such as neurotrophin-3, -4, -5, or -6), gelsolin, glucagon, kallikreins, mullerian-inhibiting substance, neurotrophic factors, p53, protein A or D, prorelaxin, relaxin A-chain, relaxin B-chain, rheumatoid factors, rhodopsin, a serum albumin (such as human serum albumin), inhibin, insulin, insulin chains, insulin A-chain, insulin D-chain, insulin receptor, proinsulin, luteinizing hormone, integrin, interleukins (ILs) (such as IL-1 to IL-10, IL-12, IL-13), erythropoietin, thrombopoietin, fibrillin, follicle stimulating hormone, clotting factors (such as factor VIIIC, factor IX, tissue factor, and von Willebrands factor), anticlotting factors (such as Protein C, atrial naturietic factor, lung surfactant), a plasminogen activator (such as human tissue plasminogen activator or urokinase), thrombin, tumor necrosis factor-D or D, D-ketoacid dehydrogenase, addressins, bone morphogenetic proteins (BMPs), collagen, colony stimulating factors (CSFs) (such as M-CSF, GM-CSF and G-CSF), decay accelerating factor, homing receptors, interferons (such as interferon-alpha, -gamma and -beta), keratin, osteoinductive factors, PRNP, regulatory proteins, superoxide dismutase, surface membrane proteins, transport proteins, T-cell receptors, antigens such as gpl 20(HIb) immuno toxins, atrial natriuretic peptide, seminal vesicle exocrine protein, D 2-microglobulin, PrP, precalcitonin, ataxin 1, ataxin 2, ataxin 3, ataxin 6, ataxin 7, huntingtin, androgen receptor, CREB-binding protein, gpl 20, p300, CREB, API, ras, NFAT, jun, fos, dentaorubral pallidoluysian atrophy-associated protein, a microbial protein (e.g., maltose binding protein, ABC transporter, glutathione S transferase, thioredoxin, D-lactamase), green fluorescent protein, red fluorescent protein, an enzyme such as superoxide dismu-tase, asparaginase, arginase, arginine deaminase, adenosine deaminase, ribonuclease, catalase, uricase, bilirubin oxidase, trypsin, papain, alkaline phosphatase, beta-glucoronidase, purine nucleoside phosphorylase or batroxobin, an opioid, e.g. endorphins, enkephalins or non-natural opioids, a hormone or neuropeptide, e.g. calcitonin, glucagon, gastrins, adreno-corticotropic hormone (ACTH), cholecystokinins, lutenizing hormone, gonadotropin-releassing hormone, chorionic gonadotropin, corticotrophin-releasing factor, vasopressin, oxytocin, antidiuretic hormones, thyroid-stimulating hormone, thyrotropin-releasing hormone, relaxin, prolactin, peptide YY, neuropeptide Y, pancreastic polypeptide, leptin, CART (cocaine and amphetamine regulated transcript), a CART related peptide, perilipin, melano-cortins (melanocyte-stimulating hormones) such as MC-4, melanin-concentrating hormones, natriuretic peptides, adrenomedullin, endothelin, secretin, amylin, vasoactive intestinal peptide (VIP), pituary adenylate cyclase activating polypeptide (PACAP), bombesin, bombesin-like peptides, thymosin, heparin-binding protein, soluble CD4, hypothalamic releasing facto-rand melanotonins or functional analogs thereof. In another embodiment of the invention the target protein may be a processing enzyme such as proteases (eg enterokinase, caspases trypsine like serine proteases), lipase, phospatase, glycosyl hydrolases (eg. mannosidases, xylosidases, fucosidases), kinase, mono or dioxidase, peroxidase, transglutaminase, carboxypeptidase, amidase, esterase, and phosphatase . . . .

Preferred sources for such mammalian polypeptides include human, bovine, equine, porcine, lupine and rodent sources, with human proteins being particularly preferred.

The biomolecule of interest of the present invention also encompasses variants of the aforementioned protein. These variants encompass, for example, protein that has the same activity as the aforementioned protein and that comprises an amino acid sequence with, in the amino acid sequence of the aforementioned protein, one or more deleted, substituted, inserted and/or added amino acids. Such protein can be exemplified by protein that has the same activity as the aforementioned protein and that comprises an amino acid sequence with, in the amino acid sequence of the aforementioned protein, one or more deleted, substituted, inserted and/or added amino acids. Two or more different types of modifications selected from deletion, substitution, insertion, and addition may be carried out concurrently.

The biomolecule of interest of the present invention also encompasses “partial peptides” of the aforementioned protein. A partial peptide of the protein can be exemplified by a partial peptide comprising an amino acid sequence in which a portion of the amino acid sequence of the aforementioned protein runs uninterrupted, wherein the partial peptide preferably has the same activity as said protein. Such a partial peptide can be exemplified by a polypeptide that has an amino acid sequence comprising at least 20 and preferably at least 50 of the amino acid residues in the amino acid sequence of the aforementioned protein. This polypeptide preferably contains the amino acid sequence that corresponds to the region that is involved with the activity of the aforementioned protein. In addition, the partial peptide used in the present invention may also be a partial peptide as yielded by a modification of this polypeptide wherein 1 or a plurality of amino acid residues (for example, approximately 1 to 20, more preferably approximately 1 to 10, and even more preferably approximately 1 to 5) is deleted from, substituted in, inserted into, and/or added to its amino acid sequence. The partial peptide used in the present invention can also be used as an antigen for antibody production.

In one embodiment of the invention, the biomolecule of interest is selected from the group comprising Human growth hormone, human insulin, follicle-stimulating hormone, Factor VIII, Erythropoeietin, Granulocyte colony-stimulating factor, Alpha-glactosidase A, Alpha-L-iduronidase, N-actetylgalactosamine-4-sulfatase, Dornase alfa, Tisssue plasminogen activator, Glucocerebrosidase, Interferon, Insulin-like growth factor 1, bovine somatotropin, Porcine somatotropin, bovine chymosin, and envelop protein of the hepaptitis B virus.

The biomolecule of interest also encompasses modified polypeptides or proteins that have underwent posttranslational and post-export modifications in the periplasm such as cyclization, glycosylation, phophorylation, methylation, oxidation, dehydratation, proteolytic cleavage.

In one embodiment, the biomolecule of interest is an enzyme for metabolizing a biomolecule in the extracellular medium (herein referred as “extracellular biomolecule”). In one embodiment, the extracellular biomolecule comprises a polysaccharide or a lipid. In one embodiment of the invention, the polysaccharide comprises alginate, pectin, cellulose, cellobiose, laminarin, or a mixture thereof. In one embodiment of the invention, the lipid comprises a fatty acid, a glycolipid, a betaine lipid, a glycerolipid, a phospholipid, a glycerolphospholipid, a sphingolipid, a sterol lipid, a prenol lipid, a saccharolipid, a polyketide, or a mixture thereof. In one embodiment of the invention, the biomolecule of interest is an enzyme converting the polysaccharide to a monosaccharide, an oligosaccharide, or both.

In one embodiment of the invention, the biomolecule of interest is an enzyme converting the lipid to a fatty acid, a monosaccharide, or both. In one embodiment of the invention, the monosaccharide or oligosaccharide is oligoalginate, mannuronate, guluronate, mannitol, a-keto acid, 4-deoxy-L-erythro-hexoselulose uronate (DEHU), 2-keto-3-deoxy D-gluconate (KDG), glucose, glucuronate, galacturonate, galactose, xylose, arabinose, or mannose. In one embodiment of the invention, the fatty acid is 14:0, trans-14, 16:0, 16:1n-7, trans-16, 16:2n-6, 18:0, 18:1n-9, 18:2n-6, 18:3n-6, 18:3n-3, 18:4n-3, 20:0, 20:2n-6, 20:3n-6, 20:4n-3,20:4n-6, or 20:5n-3.

In one embodiment of the invention, the biomolecule of interest is an enzyme converting the extracellular biomolecule to a commodity chemical. In one embodiment of the invention, the commodity chemical is ethanol, butanol, or biodiesel. In one embodiment of the invention, the biodiesel is a fatty acid, a fatty acid ester, or a terpenoid.

As used herein, the term “inactivated” refers to the interruption or to the suppression of the expression of a gene at transcriptional or translational levels. Preferably, the term “inactivated” refers to a gene whose transcription is suppressed.

According to the invention, the inactivation of a gene may be due to the mutation of the gene or to the insertion of the expression system within the coding sequence of the gene.

In the meaning of the present invention, the term “mutation” refers to a stable change in the genetic sequence. Examples of mutation which could lead to the inactivation of a gene in the present invention include, but are not limited to, point mutations, insertions, deletions and amplification or gene duplication.

Preferably, the mutation is a deletion. The term “deletion” as used herein means the loss or absence of a gene, preferably the total loss or absence of a gene. More preferably, the deletion starts at or before the start codon of the deleted gene, and ends at or after the stop codon of the deleted gene.

In one embodiment of the invention, the S gene is inactivated. The S gene encodes both a holing (S105) and an anti-holin (S107) protein. The holin protein triggers the formation of holes in the membrane. The holin is required for release of the endolysin encoded by the R gene. At the opposite, the antiholin protein inhibits the S105 hole formation. According to an embodiment, the S gene has the sequence SEQ ID NO: 1. In another embodiment, the sequence of the S gene presents a sequence identity of at least 70%, at least 75%, at least 80%, at least 85%, at least 90%, at least 91%, at least 92%, at least 93%, at least 94%, at least 95%, 5 at least 96%, at least 97%, at least 98%, and even more preferably of at least 99% with SEQ ID NO: 1.

The S gene may contain conservative sequence modifications that refer to amino acid modifications that do not significantly affect or alter the function of the S protein. Such conservative modifications include amino acid substitutions, additions and deletions. Modifications can be introduced into the S gene sequence by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. The modified sequence of the S protein may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine). Thus, one or more amino acid residues within the sequence of the S protein can be replaced with other amino acid residues from the same side chain family and the modified S protein can be tested for retained function (i.e., the properties set forth herein) by comparison with the S protein encoded by the sequence SEQ ID NO: 1.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics 5 and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

In another embodiment, the R gene is inactivated. The R protein is an endolysin: this transglycosylase degrades the murein of the cell wall of the host cell. According to an embodiment, the R gene has the sequence SEQ ID NO: 2. In another embodiment, the sequence of the R gene presents a sequence identity of at least 70%, at least 75%, at least 80%, at least 85%, at least 90%, at least 91%, at least 92%, at least 93%, at least 94%, at least 95%, 5 at least 96%, at least 97%, at least 98%, and even more preferably of at least 99% with SEQ ID NO: 2.

The R gene may contain conservative sequence modifications as described here above that refer to amino acid modifications that do not significantly affect or alter the function of the R protein. The modified sequence of the R protein may comprise one, two, three, four or more amino acid insertions, deletions or substitutions. Thus, one or more amino acid residues within the sequence of the R protein can be replaced with other amino acid residues from the same side chain family and the modified R protein can be tested for retained function (i.e., the properties set forth herein) by comparison with the R protein encoded by the sequence SEQ ID NO: 2.

In another embodiment, the Rz gene is inactivated. The Rz protein belongs to the spanin family. This protein may be involved in disrupting the outer membrane of the host cell during the lytic phase. According to an embodiment, the Rz gene has the sequence SEQ ID NO: 3. In another embodiment, the sequence of the Rz gene presents a sequence identity of at least 70%, at least 75%, at least 80%, at least 85%, at least 90%, at least 91%, at least 92%, at least 93%, at least 94%, at least 95%, 5 at least 96%, at least 97%, at least 98%, and even more preferably of at least 99% with SEQ ID NO: 3.

The Rz gene may contain conservative sequence modifications as described here above that refer to amino acid modifications that do not significantly affect or alter the function of the Rz protein. The modified sequence of the Rz protein may comprise one, two, three, four or more amino acid insertions, deletions or substitutions. Thus, one or more amino acid residues within the sequence of the Rz protein can be replaced with other amino acid residues from the same side chain family and the modified Rz protein can be tested for retained function (i.e., the properties set forth herein) by comparison with the Rz protein encoded by the sequence SEQ ID NO: 3.

In another embodiment, the Q gene is inactivated. The Q protein is a late gene regulator: the Q protein is an antiterminator of RNA synthesis from the promoter used for transcription of the entire “late” region of lambda DNA. According to an embodiment, the Q gene has the sequence SEQ ID NO: 35. In another embodiment, the sequence of the Q gene presents a sequence identity of at least 70%, at least 75%, at least 80%, at least 85%, at least 90%, at least 91%, at least 92%, at least 93%, at least 94%, at least 95%, 5 at least 96%, at least 97%, at least 98%, and even more preferably of at least 99% with SEQ ID NO: 35.

The Q gene may contain conservative sequence modifications as described here above that refer to amino acid modifications that do not significantly affect or alter the function of the Q protein. The modified sequence of the Q protein may comprise one, two, three, four or more amino acid insertions, deletions or substitutions. Thus, one or more amino acid residues within the sequence of the Q protein can be replaced with other amino acid residues from the same side chain family and the modified Q protein can be tested for retained function (i.e., the properties set forth herein) by comparison with the Q protein encoded by the sequence SEQ ID NO: 35.

In another embodiment, a nucleic acid fragment comprising the coding sequence of the S gene is deleted. According to the invention, said nucleic acid fragment does not comprise the coding sequences of the ral gene (SEQ ID NO: 4) and/or of the N gene (SEQ ID NO: 5). Therefore, according to an embodiment, when the phage is lambda, the region between residues 33930 and 35582 is not deleted.

According to another embodiment, when the phage is lambda(DE3), the region between residues 10813 and 11391 is not deleted.

Preferably, said nucleic acid fragment does not comprise the coding sequence of N and C2 (SEQ ID NO: 6), as well as the regulatory sequences of the promoter of C2. To make sure that the regulatory sequences of C2 are not deleted, the fragment to be deleted may begin at the start codon ATG of the following gene: the Cro gene. More preferably, said nucleic acid fragment does not comprise the coding sequence of ral, N and C2 (SEQ ID NO: 6), as well as the regulatory sequences of the promoter of C2.

The C2 gene encodes a repressor protein, which is important for maintaining the lysogenic state. This gene is also called cI in the sequence of several other lambdoid phages. In the lambda phage, the coding sequence of the ral, N and cI genes is from residues 33930 to 38040 or from residues 33930 to 38022. In the lambda DE3 phage, the coding sequence of the ral, N, C2 genes is from residues 10813 to 12436.

In one embodiment, the length of said deleted nucleic acid fragment is from 30 kb to 300 b, preferably from 5 kb to 500 b. In another embodiment, the length of said deleted nucleic acid fragment is 30 kb, 25 kb, 20 kb, 15 kb, 10 kb, 9 kb, 8 kb, 7 kb, 6 kb, 5 kb, 4 kb, 3 kb, 2 kb, 1 kb, 750 b, 500 b.

According to one embodiment where the phage is lambda, the deleted nucleic acid fragment starts at a position ranging from the position 35582 to the position 45186, preferably from 38041 to 45186 or from 38023 to 45186 and ends at a position ranging from the position 45510 to the position 48502

According to another embodiment where the phage is lambda DE3, the deleted nucleic acid fragment starts at a position ranging from the position 11392 to the position 16764, preferably from 12437 to the position 16764 and ends at a position ranging from 17088 and 42925.

In another embodiment, the nucleic acid fragment to be deleted comprises at least the coding sequence of the S and R genes. According to one embodiment, when the phage is lambda, the nucleic acid fragment starts at a position ranging from the position 35582 to the position 45186, preferably from 38041 to 45186 or from 38023 to 45186, and ends at a position ranging from the position 45970 to the position 48502. In another embodiment, when the phage is lambda DE3, the nucleic acid fragment starts at a position ranging from the position 11392 to the position 16764, preferably from 12437 to the position 16764, and ends at a position ranging from 17548 and 42925.

In another embodiment, the nucleic acid fragment to be deleted comprises at least the coding sequence of the S, R and Rz genes. According to one embodiment, when the phage is lambda, the nucleic acid fragment starts at a position ranging from the position 35582 to the position 45186, preferably from 38041 to 45186 or from 38023 to 45186. According to this embodiment, the fragment may end at a position ranging from the position 46428 to the position 46458. Still according to this embodiment, the nucleic acid fragment may end at a position ranging from 46428 to 48502.

According to another embodiment, when the phage is lambda DE3, the nucleic acid fragment starts at a position ranging from the position 11392 to the position 16764, preferably from 12437 to the position 16764. According to this embodiment, the fragment may end at a position ranging from position 18006 and 18036. Still according to this embodiment, the nucleic acid fragment may end at a position ranging from position 18006 to 24496. Still according to this embodiment, the nucleic acid fragment may end at a position ranging from position 18006 to 42925.

According to another embodiment, the nucleic acid fragment to be deleted comprises at least the coding sequence of the R gene.

According to one embodiment, when the phage is lambda, the nucleic acid fragment starts at a position ranging from the position 35582 to the position 45493, preferably ranging from the position 38041 or 38022 to the position 45493. According to this embodiment, the nucleic acid fragment to be deleted may end at a position ranging from 45970 to the position 48502.

According to another embodiment, when the phage is lambda DE3, the nucleic acid fragment starts at a position ranging from the position 11392 to the position 17071, preferably ranging from the position 12437 to the position 17071. According to this embodiment, the nucleic acid fragment to be deleted may end at a position ranging from 17548 to the position 24496. Still according to this embodiment, the nucleic acid fragment to be deleted may end at a position ranging from 17548 to the position 42925.

According to another embodiment, the nucleic acid fragment to be deleted comprises at least the coding sequence of the R and Rz genes.

According to one embodiment, when the phage is lambda, the nucleic acid fragment starts at a position ranging from the position 35582 to the position 45493, preferably ranging from the position 38041 or 38022 to the position 45493. According to this embodiment, the nucleic acid fragment to be deleted may end at a position ranging from 46428 to the position 46458. Still according to this embodiment, the nucleic acid fragment to be deleted may end at a position ranging from 46428 to the position 48502.

According to another embodiment, when the phage is lambda DE3, the nucleic acid fragment starts at a position ranging from the position 11392 to the position 17071, preferably ranging from the position 12437 to the position 17071. According to this embodiment, the nucleic acid fragment to be deleted may end at a position ranging from 18006 to the position 18330. Still according to this embodiment, the nucleic acid fragment to be deleted may end at a position ranging from 18006 to the position 24496. Still according to this embodiment, the nucleic acid fragment to be deleted may end at a position ranging from 18006 to the position 42925.

According to another embodiment, the nucleic acid fragment to be deleted comprises at least the coding sequence of the Q gene.

According to one embodiment where the phage is lambda, the deleted nucleic acid fragment starts at a position ranging from the position 35582 to the position 43886, preferably from 38041 to 43886 or from 38023 to 43886 and ends at a position ranging from the position 44510 to the position 48502.

According to another embodiment where the phage is lambda DE3, the deleted nucleic acid fragment starts at a position ranging from the position 11392 to the position 15464, preferably from 12437 to the position 15464 and ends at a position ranging from 16088 and 42925.

According to another embodiment, the nucleic acid fragment to be deleted comprises at least the coding sequence of the Q and S genes.

According to one embodiment where the phage is lambda, the deleted nucleic acid fragment starts at a position ranging from the position 35582 to the position 43886, preferably from 38041 to 43886 or from 38023 to 43886 and ends at a position ranging from the position 45510 to the position 48502.

According to another embodiment where the phage is lambda DE3, the deleted nucleic acid fragment starts at a position ranging from the position 11392 to the position 15464, preferably from 12437 to the position 15464 and ends at a position ranging from 17088 and 42925.

According to another embodiment, the nucleic acid fragment to be deleted comprises at least the coding sequence of the Q, S and R genes.

According to one embodiment where the phage is lambda, the deleted nucleic acid fragment starts at a position ranging from the position 35582 to the position 43886, preferably from 38041 to 43886 or from 38023 to 43886 and ends at a position ranging from the position 45970 to the position 48502.

According to another embodiment where the phage is lambda DE3, the deleted nucleic acid fragment starts at a position ranging from the position 11392 to the position 15464, preferably from 12437 to the position 15464 and ends at a position ranging from 17548 and 42925.

According to another embodiment, the nucleic acid fragment to be deleted comprises at least the coding sequence of the Q, S, R and Rz genes.

According to one embodiment where the phage is lambda, the deleted nucleic acid fragment starts at a position ranging from the position 35582 to the position 43886, preferably from 38041 to 43886 or from 38023 to 43886 and ends at a position ranging from the position 46428 to the position 48502.

According to another embodiment where the phage is lambda DE3, the deleted nucleic acid fragment starts at a position ranging from the position 11392 to the position 15464, preferably from 12437 to the position 15464 and ends at a position ranging from 18006 and 42925.

In one embodiment, the Int gene is inactivated. The Int protein manages the insertion and the excision of phage genome into the host's genome. According to an embodiment, the Int gene has the sequence SEQ ID NO: 7. In another embodiment, the sequence of the Int gene presents a sequence identity of at least 70%, at least 75%, at least 80%, at least 85%, at least 90%, at least 91%, at least 92%, at least 93%, at least 94%, at least 95%, 5 at least 96%, at least 97%, at least 98%, and even more preferably of at least 99% with SEQ ID NO: 7.

The Int gene may contain conservative sequence modifications as described here above that refer to amino acid modifications that do not significantly affect or alter the function of the Int protein. The modified sequence of the Int protein may comprise one, two, three, four or more amino acid insertions, deletions or substitutions. Thus, one or more amino acid residues within the sequence of the Int protein can be replaced with other amino acid residues from the same side chain family and the modified Int protein can be tested for retained function (i.e., the properties set forth herein) by comparison with the Int protein encoded by the sequence SEQ ID NO: 7.

In one embodiment, the Xis gene is inactivated. The Xis protein is an excisionase, which is involved in the process of excision of the lambda phage DNA out of the bacterial host chromosome. According to an embodiment, the Xis gene has the sequence SEQ ID NO: 8. In another embodiment, the sequence of the Xis gene presents a sequence identity of at least 70%, at least 75%, at least 80%, at least 85%, at least 90%, at least 91%, at least 92%, at least 93%, at least 94%, at least 95%, 5 at least 96%, at least 97%, at least 98%, and even more preferably of at least 99% with SEQ ID NO: 8.

The Xis gene may contain conservative sequence modifications as described here above that refer to amino acid modifications that do not significantly affect or alter the function of the Xis protein. The modified sequence of the Xis protein may comprise one, two, three, four or more amino acid insertions, deletions or substitutions. Thus, one or more amino acid residues within the sequence of the Xis protein can be replaced with other amino acid residues from the same side chain family and the modified Xis protein can be tested for retained function (i.e., the properties set forth herein) by comparison with the Xis protein encoded by the sequence SEQ ID NO: 8.

In one embodiment, a nucleic acid fragment comprising the coding sequence of the Int gene is deleted. According to an embodiment, the phage is lambda and the nucleic acid fragment to be deleted comprises the residues from position 27812 to 28882. According to another embodiment, the phage is lambda and the nucleic acid fragment to be deleted starts at a position ranging from position 1 to position 27812; and ends at a position ranging from 28882 to 33929.

In one embodiment, a nucleic acid fragment comprising the coding sequence of the Xis gene is deleted.

According to one embodiment, the phage is lambda and the deleted nucleic acid fragment to be deleted comprises the residues from position 28860 to 29078. According to another embodiment, the phage is lambda and the fragment starts at a position ranging from position 1 to position 28860, and ends at a position ranging from position 29078 to position 33929.

According to another embodiment, the phage is DE3 and the deleted nucleic acid comprises the residues from position 5586 to 5804. According to another embodiment, the fragment to be deleted starts at a position ranging from position 1 to position 5586, and ends at a position ranging from position 5804 to position 10812.

In one embodiment, a nucleic acid fragment comprising the coding sequences of the Xis and Int genes is deleted. According to an embodiment, the phage is lambda and the nucleic acid fragment to be deleted comprises the residues from position 27812 to 29078. According to another embodiment, the phage is lambda and the nucleic acid fragment to be deleted starts at a position ranging from position 1 to position 27812; and ends at a position ranging from 29078 to 33929.

In one embodiment of the invention, the genetically modified phage of the invention is further inactivated for the kil gene. According to an embodiment, the kil gene has the sequence SEQ ID NO: 36). In another embodiment, the kil gene presents a sequence identity of at least 70, 75, 80, 85, 90, 91, 92, 93, 94, 95, 96, 97, 98, more preferably of at least 99% with SEQ ID NO: 36. Preferably, the coding sequence of the kil gene is deleted.

According to one embodiment, the phage is lambda and the deleted nucleic acid fragment comprises the residues from position 33187 to 33331.

According to another embodiment, the phage is DE3 and the deleted nucleic acid comprises the residues from position 9913 to 10057.

According to an embodiment, the attP sequence is not deleted from the sequence of the phage of the invention. The attP sequence is SEQ ID NO: 9. The attP sequence is located from position 27586 and 27817 in the sequence of the Lambda Phage, and from position 42788 to 42925 and from 1 to 94 in the genome of the Lambda DE3 phage (the genome of the phage is circular, the attP sequence is thus continue).

In one embodiment of the invention, the genetically modified phage comprises the attP sequence and the sequence of the C2 gene. In another embodiment, the genetically modified phage consists of the attP sequence and the sequence of the C2 gene.

According to a preferred embodiment, one genetically modified phage of the invention P11 has the sequence SEQ ID NO: 10 and is (DE3) ΔS-C, Δxis-ea10 (DE3 refers to lambda phage DE3 wherein the T7 RNA polymerase gene has been integrated within the sequence of int gene). Said modified phage P11 corresponds to the sequence NC_{—}001416 wherein the coding sequences of genes S, R, Rz, Xis and Int are deleted.

According to another embodiment, one genetically modified phage of the invention P12 corresponds to the sequence NC_{—}001416 wherein the coding sequences of genes Int and S are deleted (one example of P12 is the sequence DE3 ΔS, Δxis-ea10).

According to another embodiment, one genetically modified phage of the invention P13 corresponds to the sequence NC_{—}001416 wherein the coding sequences of genes S, R, Rz, Q, Xis and Int are deleted (one example of P13 is the sequence DE3 ΔS-C, Δxis-ea10, ΔQ).

According to another embodiment, one genetically modified phage of the invention P14 corresponds to the sequence NC_{—}001416 wherein the coding sequences of genes R and Int are deleted (one example of P14 is the sequence DE3 ΔR).

According to another embodiment, one genetically modified phage of the invention P15 corresponds to the sequence NC_{—}001416 wherein the coding sequences of genes Q and Int are deleted (one example of P15 is the sequence DE3 ΔQ).

According to another embodiment, one genetically modified phage of the invention P16 corresponds to the sequence NC_{—}001416 wherein the coding sequences of genes S and Xis are deleted (one example of P16 is the sequence NC_{—}001416 ΔS, Δxis-ea10).

According to another embodiment, one genetically modified phage of the invention P17 corresponds to the sequence NC_{—}001416 wherein the coding sequences of genes R and Xis are deleted (one example of P17 is the sequence NC_{—}001416 ΔR, Δxis-ea10).

According to another embodiment, one genetically modified phage of the invention P18 corresponds to the sequence NC_{—}001416 wherein the coding sequences of genes Q and Xis are deleted (one example of P18 is the sequence NC_{—}001416 Δxis-ea10, ΔQ).

According to another embodiment, one genetically modified phage of the invention p19 corresponds to the sequence NC_{—}001416 wherein the coding sequences of genes R, Rz and Int are deleted (one example of P19 is the sequence DE3 ΔR, ΔRz).

According to another embodiment, one genetically modified phage of the invention P20 corresponds to the sequence NC_{—}001416 wherein the coding sequences of genes S, Rz and Int are deleted (one example of P20 is the sequence DE3 ΔS, ΔRz).

According to another embodiment, one genetically modified phage of the invention P21 corresponds to the sequence NC_{—}001416 wherein the coding sequences of genes Rz, Q and Int are deleted (one example of P21 is the sequence DE3 ΔRz, ΔQ).

According to another embodiment, one genetically modified phage of the invention P22 corresponds to the sequence NC_{—}001416 wherein the coding sequences of genes S, Q and Int are deleted (one example of P22 is the sequence DE3 ΔS, ΔQ).

According to another embodiment, one genetically modified phage of the invention P23 corresponds to the sequence NC_{—}001416 wherein the coding sequences of genes R, Q, and Int are deleted (one example of P23 is the sequence DE3 ΔR, ΔQ).

According to another embodiment, one genetically modified phage of the invention P24 corresponds to the sequence NC_{—}001416 wherein the coding sequences of genes S, R and Int are deleted (one example of P24 is the sequence DE3 ΔS, ΔR).

According to another embodiment, one genetically modified phage of the invention P25 corresponds to the sequence NC_{—}001416 wherein the coding sequences of genes R, Rz and Xis are deleted (one example of P25 is the sequence NC_{—}001416 ΔR, Δxis-ea10, ΔRz).

According to another embodiment, one genetically modified phage of the invention P26 corresponds to the sequence NC_{—}001416 wherein the coding sequences of genes S, Rz and Xis are deleted (one example of P26 is the sequence NC_{—}001416 ΔS, Δxis-ea10, ΔRz).

According to another embodiment, one genetically modified phage of the invention P27 corresponds to the sequence NC_{—}001416 wherein the coding sequences of genes Q, Rz and Xis are deleted (one example of P27 is the sequence NC_{—}001416 ΔRz, Δxis-ea10, ΔQ).

According to another embodiment, one genetically modified phage of the invention P28 corresponds to the sequence NC_{—}001416 wherein the coding sequences of genes S, Q and Xis are deleted (one example of P28 is the sequence NC_{—}001416 ΔS, Δxis-ea10, ΔQ).

According to another embodiment, one genetically modified phage of the invention P29 corresponds to the sequence NC_{—}001416 wherein the coding sequences of genes R, Q and Xis are deleted (one example of P29 is the sequence NC_{—}001416 ΔR, Δxis-ea10, ΔQ).

According to another embodiment, one genetically modified phage of the invention P30 corresponds to the sequence NC_{—}001416 wherein the coding sequences of genes R, S and Xis are deleted (one example of P30 is the sequence NC_{—}001416 ΔS, Δxis-ea10, ΔR).

According to another embodiment, one genetically modified phage of the invention P31 corresponds to the sequence NC_{—}001416 wherein the coding sequences of genes R, Xis and Int are deleted (one example of P31 is the sequence DE3 ΔR, Δxis-ea10).

According to another embodiment, one genetically modified phage of the invention P32 corresponds to the sequence NC_{—}001416 wherein the coding sequences of genes S, Xis and Int are deleted (one example of P32 is the sequence DE3 ΔS, Δxis-ea10).

According to another embodiment, one genetically modified phage of the invention P33 corresponds to the sequence NC_{—}001416 wherein the coding sequences of genes Q, Xis and Int are deleted (one example of P33 is the sequence DE3 Δxis-ea10, ΔQ).

According to another embodiment, one genetically modified phage of the invention P34 corresponds to the sequence NC_{—}001416 wherein the coding sequences of genes S, R, Xis and Int are deleted (one example of P34 is the sequence DE3 ΔS, Δxis-ea10, ΔR).

According to another embodiment, one genetically modified phage of the invention P35 corresponds to the sequence NC_{—}001416 wherein the coding sequences of genes R, Q, Xis and Int are deleted (one example of P35 is the sequence DE3 ΔR, Δxis-ea10, ΔQ).

According to another embodiment, one genetically modified phage of the invention P36 corresponds to the sequence NC_{—}001416 wherein the coding sequences of genes S, Q, Xis and Int are deleted (one example of P36 is the sequence DE3 ΔS, Δxis-ea10, ΔQ).

According to another embodiment, one genetically modified phage of the invention P37 corresponds to the sequence NC_{—}001416 wherein the coding sequences of genes R, Rz, Xis and Int are deleted (one example of P37 is the sequence DE3 ΔR, Δxis-ea10, ΔRz).

According to another embodiment, one genetically modified phage of the invention P38 corresponds to the sequence NC_{—}001416 wherein the coding sequences of genes S, Rz, Xis and Int are deleted (one example of P38 is the sequence DE3 ΔS, Δxis-ea10, ΔRz).

According to another embodiment, one genetically modified phage of the invention P39 corresponds to the sequence NC_{—}001416 wherein the coding sequences of genes Rz, Q, Xis and Int are deleted (one example of P39 is the sequence DE3 ΔRz, Δxis-ea10, ΔQ).

According to another embodiment, one genetically modified phage of the invention P40 corresponds to the sequence NC_{—}001416 wherein the coding sequences of genes S, R, Q and Int are deleted (one example of P40 is the sequence DE3 ΔS, ΔR, ΔQ).

According to another embodiment, one genetically modified phage of the invention P41 corresponds to the sequence NC_{—}001416 wherein the coding sequences of genes S, R, Rz and Int are deleted (one example of P41 is the sequence DE3 ΔS-C).

According to another embodiment, one genetically modified phage of the invention P42 corresponds to the sequence NC_{—}001416 wherein the coding sequences of genes R, Rz, Q and Int are deleted (one example of P42 is the sequence DE3 ΔR, ΔRz, ΔQ).

According to another embodiment, one genetically modified phage of the invention P43 corresponds to the sequence NC_{—}001416 wherein the coding sequences of genes S, Rz, Q and Int are deleted (one example of P43 is the sequence DE3 ΔS, ΔRz, ΔQ).

According to another embodiment, one genetically modified phage of the invention P44 corresponds to the sequence NC_{—}001416 wherein the coding sequences of genes S, R, Q and Xis are deleted (one example of P44 is the sequence NC_{—}001416 ΔS, ΔR, Δxis-ea10, ΔQ).

According to another embodiment, one genetically modified phage of the invention P45 corresponds to the sequence NC_{—}001416 wherein the coding sequences of genes S, R, Rz and Xis are deleted (one example of P45 is the sequence NC_{—}001416 ΔS-C, Δxis-ea 10).

According to another embodiment, one genetically modified phage of the invention P46 corresponds to the sequence NC_{—}001416 wherein the coding sequences of genes R, Rz, Q and Xis are deleted (one example of P46 is the sequence NC_{—}001416 ΔR, ΔRz, Δxis-ea10, ΔQ).

According to another embodiment, one genetically modified phage of the invention P47 corresponds to the sequence NC_{—}001416 wherein the coding sequences of genes S, Rz, Q and Xis are deleted (one example of P47 is the sequence NC_{—}001416 ΔS, ΔRz, Δxis-ea10, ΔQ).

According to another embodiment, one genetically modified phage of the invention P48 corresponds to the sequence NC_{—}001416 wherein the coding sequences of genes S, R, Q, Xis and Int are deleted (one example of P48 is the sequence DE3 ΔS, ΔR, Δxis-ea10, ΔQ).

According to another embodiment, one genetically modified phage of the invention P49 corresponds to the sequence NC_{—}001416 wherein the coding sequences of genes S, Rz, Q, Xis and Int are deleted (one example of P49 is the sequence DE3 ΔS, ΔRz, Δxis-ea10, ΔQ).

According to another embodiment, one genetically modified phage of the invention P50 corresponds to the sequence NC_{—}001416 wherein the coding sequences of genes R, Rz, Q, Xis and Int are deleted (one example of P50 is the sequence DE3 ΔR, ΔRz, Δxis-ea10, ΔQ).

According to another embodiment, one genetically modified phage of the invention P51 corresponds to the sequence NC_{—}001416 wherein the coding sequences of genes S, R, Q, Rz and Int are deleted (one example of P51 is the sequence DE3 ΔS-C, ΔQ).

According to another embodiment, one genetically modified phage of the invention P52 corresponds to the sequence NC_{—}001416 wherein the coding sequences of genes S, R, Rz, Q and Xis are deleted (one example of P52 is the sequence NC_{—}001416 ΔS-C, Δxis-ea10, ΔQ).

According to another embodiment, one genetically modified phage of the invention P53 corresponds to the sequence NC_{—}001415 wherein the coding sequences of genes Q, Xis and Int are deleted (one example of P53 is the sequence DE3 Δxis-ea10, ΔQ).

The present invention also relates to a process for producing the modified phage of the invention, wherein said process comprises at least two steps of deletion of genes.

In an embodiment, the process of the invention is carried out with a phage integrated in the genome of a host cell.

In an embodiment, the host cell is a microorganism, preferably a prokaryote, more preferably a bacterium, more preferably a gram negative bacterium. Advantageously, the host cell is a bacterium from the Enterobacteriacea family according to the current applicable taxonomy. If the taxonomy should change, the skilled artisan would know how to adapt the changes in the taxonomy to deduce the strains that could be used in the present invention. Examples of bacteria from the Enterobacteriacea family include, but are not limited to, bacteria belonging to the genera Escherichia, Enterobacter, Erwinia, Klebsiella, Pantoea, Photorhabdus, Providencia, Salmonella, Serratia, Shigella, Morganella and Yersinia. According to a preferred embodiment, the host cell belongs to the Escherichia genus, and more preferably the host cell is Escherichia coli (E. coli).

Methods for deleting genes from an integrated phage are well known to the skilled artisan. Examples of such methods include, but are not limited to homologous recombination (also called recombineering) using the lambda Red-encoded genes: exo, bet and gam. It is also possible to use the corresponding recE and recT genes from the prophage Rac. The genes exo and recE encode a 5′-3′ exonuclease that produces 3′ overhangs. The bet and recT genes encode a pairing or also called annealing protein that binds the 3′ overhangs and mediates its annealing and homologous recombination between two complementary DNA sequences. The gam gene encodes an inhibitor of the E. coli RecBCD exonuclease and thus protects linear DNA fragments of interest. The method of recombineering was well described by several researchers including Datsenko and Wanner (PNAS 97-12, 6640-6645, 2000) and Stewart et al (WO0104288). The principle of the method is to generate (by PCR amplification for example) a DNA fragment containing the fragment to integrate and two recombination arms. These arms are homologous to the regions adjacent to the gene to be inactivated. They will be used to target the insertion of the fragment of interest. It is possible to create this kind of DNA fragment by PCR using primers containing homologous arms from 20 to 60 nucleotides.

The present invention also relates to a host cell comprising the genetically modified phage of the invention. In a preferred embodiment, the host cell of the invention comprises the genetically modified phage integrated in its genome.

In one embodiment, the host cell is a microorganism, preferably a prokaryote, more preferably a bacterium, more preferably a gram negative bacterium. Advantageously, the host cell is a bacterium from the Enterobacteriacea family according to the current applicable taxonomy. If the taxonomy should change, the skilled artisan would know how to adapt the changes in the taxonomy to deduce the strains that could be used in the present invention. Examples of bacteria from the Enterobacteriacea family include, but are not limited to, bacteria belonging to the genera Escherichia, Enterobacter, Erwinia, Klebsiella, Pantoea, Photorhabdus, Providencia, Salmonella, Serratia, Shigella, Morganella and Yersinia. According to a preferred embodiment, the host cell belongs to the Escherichia genus, and more preferably the host cell is Escherichia coli (E. coli). Examples of strains of E. coli which could be used in the present invention include, but are not limited to, strains derived from E. Coli K-12, E. coli B or E. coli W, such as, for example, MG1655, W3110, DG1, DG2, Top10, DH10B, DH5alpha, HMS174, BL21, BL21(DE3), HMS174(DE3), BL21(DE3) pLysS, BL21(DE3) pLysE.

In one embodiment, genes of the host cells may be inactivated.

In one embodiment, the gene tonA (also known as fhuA, SEQ ID NO: 11) is inactivated. The TonA/FhuA protein is a receptor for the phages Ti, T5 and Phi80.

In one embodiment, the gene galK (SEQ ID NO: 12) is inactivated. The deletion of this gene allows the use of the galK positive/negative selection for deletion of genes by a method based on homologous recombination.

In one embodiment, the gene araB (SEQ ID NO: 13) is inactivated. In another embodiment, the gene araA (SEQ ID NO: 14) is inactivated. The inactivation of araB and/or araA is recommended for the use of the promoter PBAD (inducible by arabinose) within the host cell.

In one embodiment, the gene lon (SEQ ID NO: 15) and/or the gene ompT (SEQ ID NO: 16) are inactivated. The Lon protein is an ATP dependent protease. The OmpT protein is an outer membrane protease. Preferably, the genes lon and ompT are inactivated.

In one embodiment, the gene rcsA (SEQ ID NO: 17) is inactivated. The protein RcsA is a positive regulator of the synthesis of the capsule, which is degraded by the Lon protease.

In one embodiment, the gene hsdR (SEQ ID NO: 18) and/or the gene mrr (SEQ ID NO: 19) are inactivated. The HsdR and Mrr proteins are restriction enzymes with different specificity. Preferably, the genes hsdR and mrr are both inactivated.

In one embodiment, the gene endA (SEQ ID NO: 20) and/or the gene recA (SEQ ID NO: 21) are inactivated. EndA is a DNA specific endonuclease. RecA is a recombination protein with protease and nuclease activity. Preferably, the genes endA and recA are both inactivated.

In one embodiment of the invention, at least one of the genes tonA, galK, araB, araA, lon, ompT, rcsA, hsdR, mrr, endA and recA are inactivated. Preferably, the inactivated genes are deleted.

In a preferred embodiment, the genes tonA, galK, araB, lon, ompT, rcsA, hsdR, mrr, endA and recA are inactivated. Preferably, the genes tonA, galK, araB, lon, ompT, rcsA, hsdR, mrr, endA and recA are deleted.

In one embodiment of the invention, the host cell of the invention is transformed with a nucleic acid sequence encoding a toxic molecule, as described in US2004/0115811. The presence of the nucleic acid sequence encoding the toxic molecule will allow the selection of recombinant clones having integrated a gene of interest and a nucleotide sequence encoding a functional antidote protein to a toxic molecule, wherein said recombinant clones are the ones which survive following their integration into a host cell comprising in its genome a nucleotide sequence encoding said toxic molecule.

According to a preferred embodiment of the present invention, the antidote protein and the toxic molecule are respectively, an anti-poison protein and a poison protein. Said anti-poison or poison proteins could be wild type proteins or modified proteins which are naturally or artificially poisonous and affect one or more vital functions of a cell (preferably, a prokaryote cell) and may lead to the killing of the cell.

The antidote protein and the toxic molecule are preferably selected from the group consisting of CcdA/CcdB proteins, Kis/Kid proteins, Phd/Doc proteins, RelB/relE proteins, PasB (or PasC)/PasA proteins, mazF/mazE proteins as described in US2004/0115811, or any other couple of anti-poison/poison molecules which are or are not of plasmid origin. The toxic molecule can also be a toxin protein being naturally or artificially toxic and affecting one or more vital functions of a (prokaryote) cell. The protein encoded by the gene sacB (from Bacillus amylolique-faciens), the protein GpE, the protein GATA-1 and the protein Crp are other examples of such toxic molecules. The gene sacB encodes the levan sucrase which catalyses the hydrolysis of sucrose into products which are toxic for E. Coli (Pierce et al. Proc. Natl. Acad. Sci., Vol. 89, N[deg.]6 (1992) p. 2056-2060). The protein GpE encodes the E genes from the bacteriophage [phi]X174 which includes six unique restriction sites and encodes gpE and which causes lysis of E. Coli cell (Heinrich et al., Gene, Vol. 42(3) (1986) p. 345-349). The protein GATA-1 has been described by Trudel et al. (Biotechniques 1996, Vol. 20(4), p. 684-693). The protein Crp has been described by Schlieper et al. (Anal. Biochem. 1998, Vol. 257(2), p. 203-209).

The antidote proteins to said toxic molecule are any protein able to reduce or suppress the effect of the corresponding toxic molecule on a cell (preferably a prokaryotic cell), when said toxic molecule is produced by said cell.

According to a preferred embodiment, the host cell of the invention comprises a nucleic acid sequence encoding the protein CcdB. The ccdB gene has the sequence SEQ ID NO: 22.

The present invention also relates to a kit comprising a host cell as hereinabove described, wherein the gene encoding a poison protein is inserted, and a plasmid carrying the nucleic acid sequence of the gene encoding the anti-poison protein. The expression of the anti-poison protein in the host cell is required for maintaining the viability of the host cell. In a preferred embodiment, the poison protein is encoded by the ccdB gene, and the anti-poison protein is encoded by the ccdA gene (SEQ ID NO: 23).

According to an embodiment of the invention, the plasmid of the kit may further contain, or may be modified to further contain, the nucleic acid sequence of the biomolecule of interest.

The present invention also refers to a process for preparing a host cell as hereinabove described.

In one embodiment, the process of the invention comprises a step of infection of the host cell by a genetically modified phage according to the invention. In one embodiment of the invention, said infection step includes the use of a helper phage. In the meaning of the present invention, the term “helper phage” refers to a phage used to complement a deletion or an inactivation of another phage. The helper phage will provide the missing functions to another phage to be able to infect bacteria or to prepare phage stock. Usually, the helper phage cannot form a lysogen by itself because it is cI minus (it has no repressor and is thus virulent).

The process for infecting a host cell with a phage using a helper phage is well known in the art. The first step is the preparation of the lysates and the second one is the lysogenization. Briefly, the bacterial lysates of the helper phage are prepared using standard methods as described in “Molecular cloning: a laboratory manual”, Sambrook et al (2001, ISBN 978-087969577-4) or in “Large- and Small-Scale Preparation of Bacteriophage lambda lysate and DNA”, Su et al, BioTechniques 25:44-46 (July 1998). Preparation of the phage of interest is done using the same principle, after phage induction (most often using UV irradiation or any situation where a lysogen undergoes DNA damage or the SOS response of the host or Cro production) in order to launch the lytic cycle and using a helper phage to provide the missing functions. An alternative to the helper phage is the use of a plasmid encoding the missing functions.

Next, the phage lysates are mixed with the targeted bacteria and plated on LB plates in order to get lysogens (as described in lambda DE3 lysogenization kit from Novagen, User Protocol TB031 or an alternative method is described in Studier and Moffat, Journal of Molecular Biology, 1986, 189:113-130). A selection phage can be used to select specifically bacteria containing the phage of interest. This selection phage is a virulent phage having the same immunity as the phage of interest. Consequently, the selection phage is unable to form plaques or to kill bacteria lysogens for the phage of interest because this phage produces the cI repressor (also called C2 in DE3 lambda phage).

The present invention thus also relates to a kit comprising the modified phage of the invention, as hereinabove described, and a helper phage. Examples of helper phages include, but are not limited to, helper phage B10 or any other lambdoid phage with a different immunity than the phage of interest (example: phage with immunity 434, 80, . . . ). Some helper phages and their manipulations were well described in the literature including in Haldimann and Wanner (Journal of Bacteriology, 183, 6384-6393, 2001).

In one embodiment, the process for preparing a host cell of the invention further comprise a step of deletion, wherein nucleic acid sequences of the host cell are deleted. Methods for deleting the sequence of a gene are well known by the skilled artisan. The more efficient method is the homologous recombination method mediated by the lambda Red-encoded genes or the recE and recT genes from the prophage Rac. As described above, this method was well described by several researchers including Datsenko and Wanner (PNAS 97-12, 6640-6645, 2000) and Stewart et al (WO0104288). PCR products are generated using primers with 20- to 60-nt extensions that are homologous to regions adjacent to the gene to be inactivated. Since only a small amount of bacteria will effectively recombine the fragment of interest, it is necessary to have a strong selection marker to select it. Antibiotic markers can be used to select the recombinants: the modified primers are used to amplify an antibiotic resistance gene. After transformation and activation of the recombination genes, recombinant bacteria are selected on medium containing the appropriate antibiotic. In this case, the targeted gene is replaced by an antibiotic resistance gene. In order to use the same strategy for the next deletion, it is necessary to remove this antibiotic resistance gene during a second step. As described in Datsenko and Wanner, it is possible to use antibiotic resistance gene that are flanked by FRT (FLP recognition target) sites. The resistance genes are then eliminated by using a helper plasmid encoding the FLP recombinase. The antibiotic resistance gene is removed by this site-specific recombinase but this method leaves traces: one site-specific recombination site is still present after removal of the antibiotic resistance gene.

To avoid the presence of this site, more preferably, the method of the invention uses galK as a marker gene. The principle of the galK selection is described in Warming et al. (Nucleic acid research, 2005, 33(4)). This method uses galK as a positive selection marker (growth on minimal medium containing galactose) during the first recombination (insertion). The removal of this marker is performed during a second homologous recombination step. During this step, galK is used as a negative selection marker on minimal medium containing 2-deoxy-galactose (DOG). The galK gene product, galactokinase, catalyzes the first step in the galactose degradation pathway. Galactokinase also efficiently catalyzes the phosphorylation of the DOG galactose analog. The product of this reaction cannot be further metabolized, leading to the accumulation of a toxic molecule (2-deoxy-galactose-1-phosphate). The advantage of this method is to avoid the presence of specific recombination site after deletion of the targeted gene and removal of the selective marker.

The present invention also relates to a process for producing a biomolecule of interest, comprising
cultivating a host cell comprising the genetically modified phage according to the invention and the nucleic acid sequence of the biomolecule of interest,
recovering the biomolecule of interest.

In one embodiment of the invention, the nucleic acid sequence of the biomolecule of interest is comprised within the expression system of the genetically modified phage. According to this embodiment, the production of the biomolecule of interest is direct, i.e. results from the expression of the gene of the expression system, for example by culture in a medium wherein the promoter comprised in the expression system is induced.

In another embodiment of the invention, the expression system of the genetically modified phage comprises the nucleic acid sequence of the T7 RNA polymerase under the control of a lac promoter, preferably the lacUV5 promoter. According to this embodiment, the process for producing the biomolecule of interest comprises the transformation of the host cell with a plasmid comprising the nucleic acid sequence of the biomolecule of interest under the control of a the T7 promoter. The expression from the T7 promoter is under the control of T7 RNA polymerase, which is stringently specific for the T7 promoter, i.e. the T7 promoter can only be utilized by the RNA polymerase of bacteriophage T7. When IPTG is added to the culture medium, T7 RNA polymerase is expressed by transcription from the lac promoter, and allows the expression of the biomolecule of interest.

In the meaning of the present invention, the term “T7 promoter” includes promoters that are present in the genome of bacteriophage T7, as well as consensus sequences and variants of such promoters with the ability to mediate transcription by the T7 RNA polymerase. The bacteriophage T7 contains seventeen different promoter sequences, all of which comprise a highly conserved nucleotide sequence.

According to a preferred embodiment, the plasmid comprising the nucleic acid sequence of the biomolecule of interest also comprises the nucleic acid sequence of ccdA, and the host cell comprises the sequence of ccdB integrated in its genome. Therefore, only recombinant clones containing the plasmid are propagated.

According to one embodiment, the biomolecule of interest is secreted by the host cell in the fermentation broth. According to this embodiment, the biomolecule of interest may be easily recovered from the fermentation broth.

According to another embodiment, the biomolecule of interest is not secreted by the host cell in the fermentation broth. Methods for recovering an intracellular biomolecule of interest are well-known in the art. Examples of such method include, but are not limited to, the use of trichloroacetic acid (TCA) or cracking buffer containing sodium dodecyl sulfate (SDS) to recover total cytoplasmic proteins in denaturing conditions or the use of sonication, French press or equivalent to disrupt bacteria under pressure in order to recover total cytoplasmic proteins in native (not denaturing) conditions. Next, the protein of interest can be purified using specific methods including but not limited to the use of affinity or ion exchange columns.

### BRIEF DESCRIPTION OF THE DRAWINGS

FIG. 1: Picture of a SDS-Page gel colored with Coomassie blue staining, showing the production of the protein of interest. 1 and 6: size; 2: pellet [pScherryl] M11DE3 before induction; 3: pellet [pScherryl] M11DE3 after induction; 4: pellet [pScherryl] HMS174DE3 before induction; 5: pellet [pScherryl] HMS174DE3 after induction. The array is for identifying the protein of interest.

### EXAMPLES

The present invention is further illustrated by the following examples.

### Example 1

### Deletion of the xis, exo, bet, gam, kil, cIII, N and ral Genes

a) Deletion of xis DNA Region and Replacement by galK

First the galK gene was amplified by polymerase chain reaction (PCR) on pGalK plasmid using the primers XisgalKstart (5′ GGGGGTAAATCCCGGCGCTCATGACTTCGCCTTCTTCCCAGAATTCCTGTTGAC AATTA 3′, SEQ ID NO: 24) and XisgalK stop (5′ GTTCTGATTATTGGAAATCTTCTTGCCCTCCAGTGTGAGCAGCACTGTCCTGCT CCTTG 3′, SEQ ID NO: 25). These primers contain at the 5′ end a sequence of 40 bases identical to the DNA target (italicized). These sequences of 40 bases are the recombination arms. The 3′ ends were designed to amplify the galK gene and its constitutive promoter. The DNA fragment amplified by PCR (1315 bp) targeted the genes xis, exo, bet, gam, kil and cIII (DNA fragment of 5133 bp): these genes were replaced during homologous recombination by the galK gene and its promoter. Electrocompetent bacteria carrying the T7(DE3) prophage and the pKD46 plasmid were prepared according to Datsenko and Wanner (PNAS 97-12, 6640-6645, 2000). Next, the amplified galK fragment was electroporated in these bacteria according to standard procedures (200 ng of DNA fragment was used for each electroporation). SOC medium was added and bacteria were incubated during 1 hour at 37° C. Next, bacteria were washed (centrifuged, medium removal, addition of fresh medium and resuspension) twice with M9 minimal medium (Sambrook et al (2001, ISBN 978-087969577-4)) and plated on bacterial plates containing minimal M9 medium and 1% galactose. Plates were incubated at 37° C. during 1 or 2 days.

The next step was the bacterial screening: PCR screening was performed directly on colonies using the Xis1 (5′GTCTTCAAGTGGAGCATCAG3′, SEQ ID NO: 26) and Xis4 (5′ACCAGGACTATCCGTATGAC3′, SEQ ID NO: 27) primers. An amplification of a 5774 bp DNA fragment corresponds to a non-modified chromosome (non-recombinant colony) and, on the contrary, an amplification of a 1955 bp DNA fragment corresponds to a recombinant chromosome. Bacteria allowing amplification of the 1955 bp DNA fragment were selected and streaked two times on selective plates (minimal M9 medium supplemented with 1% galactose) in order to purify it and to remove possible unmodified copies of the chromosome. The PCR screening was done one more time at the end of the purification step and 3 bacteria allowing amplification of the 1955 bp DNA fragment were selected. The amplified DNA fragments corresponding to these bacteria were sequenced using the same primers in order to confirm the DNA recombination and the deletion of the Xis DNA region (xis, exo, bet, gam, kil and cIII genes).

b) GalK Removal

A DNA fragment containing large recombination arms (of 350 bp and 343 bp) was constructed by PCR to remove galK and the N and ral genes. The first arm was amplified byPCR on bacterial colonies containing the T7(DE3) prophage using the Xis1 and Xis2 (5′CCAAACGGAACAGATGAAGAAGGCGAAGTCATGAG3′, SEQ ID NO: 28) primers. A DNA fragment of 365 bases was amplified. The second arm was also performed by PCR on the same bacteria using the Xis3 (5′ GACTTCGCCTTCTTCATCTGTTCCGTTTGGCTTCC3′, SEQ ID NO: 29) and Xis6 (5′ GTAATGGAAAGCTGGTAGTCG3′, SEQ ID NO: 30) primers. A DNA of fragment of 358 bases was amplified. Both recombination arms were purified after agarose gel electrophoresis. Xis2 and Xis3 primers were designed to generate DNA fragments containing an identical sequence of 30 base pairs. This sequence was used to join both recombination arms in a third PCR using Xis1 and Xis6 primers. A DNA fragment of 693 bp was generated. This DNA fragment was electroporated in bacteria selected above and carrying the pKD46 plasmid (prepared as described in Datsenko and Wanner). SOC medium was added and bacteria were incubated at 37° C. during 1 hour. Next, bacteria were washed twice with M9 medium and plated on selective plates containing M9 medium supplemented with 0.2% glycerol and 1% DOG. Plates were incubated during 2 days at 37° C. Several colonies were screened by PCR using the Xis5 (5′ CAGCCGTAAGTCTTGATCTC3′, SEQ ID NO: 31) and Xis7 (5′CAGCAGGCATGATCCAAGAG3′, SEQ ID NO: 32) primers. An amplification of 3246 bp corresponding to the unmodified DNA chromosome was always obtained instead of an amplification of 1122 bp corresponding to the modified chromosome. The experiment was reproduced completely and independently three times without success: no bacteria comprising the desired deletion was obtained.

Consequently, we decided to remove only the GalK fragment and to leave the N and ral genes. The DNA fragment containing the recombination arms was generated as described above using Xis,1 Xis2b (5′ TTTGCCCTCCAGTGTGAAGAAGGCGAAGTCATGAG3′, SEQ ID NO: 33) for the first recombination arm (365 bp) and Xis8 (5′ CTCATGACTTCGCCTTCTTCACACTGGAGGGCAAAGAAG, SEQ ID NO: 34) and Xis4 for the second recombination arm (384 bp). The joining PCR was performed using Xis 1 and Xis4 primers and generated a DNA fragment of 714 bp. This fragment was electroporated as described above in the bacteria selected and carrying the pKD46 plasmid. Bacteria were plated on the same selective plates containing DOG and incubated during two days at 37° C. PCR screening was performed using Xis5 and Xis6 primers. Bacteria showing an amplification of 1770 bp (instead of an amplification of 3010 bp corresponding to the unmodified chromosome) were selected and purified. The DNA fragment was sequenced using the Xis5 and Xis6 primers and showed the right removal of the galK gene. This recombination was performed only once since bacteria were obtained immediately.

These results show that it was not possible to remove galK associated to the N and ral genes using homologous recombination. However, using exactly the same procedure, we were able to remove galK alone.

In conclusion, we demonstrated that removal of the N and ral genes leads to the death of bacteria, which was unexpected.

### Example 2

### Protein Expression Using MG1655

In order to test the protein production efficiency of the strain constructed according to the invention, small-scale expression test were performed using bacteria called M11(DE3). The genotype of this strain is MG1655 ΔgalK, ΔrcsA, Δlon, ΔhsdR-mrr, ΔfhuA, ΔendA, ΔrecA, ΔaraB, ΔompT, λ-DE3 (T7pol, Δxis-ea10, ΔS-C). Since MG1655 is an Escherichia coli K-12, it was compared to another K-12 strain used as a standard in the field of protein production and called HSM174(DE3) (genotype: recA1, hsdR, λ-DE3, (Rif R)). Both strains were transformed with pSCherryl plasmid DNA (Delphi Genetics, Belgium). This plasmid encodes a protein called “cherry” easily detectable (by eyes, red color) under the control of the T7 promoter.

Protocol for a small-scale expression using IPTG:

1) Two Erlenmeyer flasks containing 10 ml of LB medium were inoculated each with a single colony of the HMS174 (DE3) and the M11(DE3) carrying both the pSCherryl plasmid and incubated at 30° C. overnight.
2) Two new flasks containing fresh medium were inoculated with 1 ml of the overnight cultures and incubated with shaking at 37° C. until OD600 reached 0.6.
3) A sample (1 ml) from each flask was taken and centrifuged. The medium was discarded and the pellet was kept on ice. The samples were the non-induced controls. To induce protein expression in the remaining culture, IPTG (Isopropyl β-D-1-thiogalactopyranoside, 90 μl of a fresh 100 mM stock solution) was added to reach a final concentration of 1 mM in both flasks. Incubation of both flasks was continued for 4 hours.
4) At the end of the induction period, the Optical Density at 600 nm was measured for each culture (1.09 for M11(DE3) and 1.13 for HMS174(DE3)). A 1 ml sample of each flask was centrigufed at maximum speed (13000 g) for 10 min at 4° C. It was observed that the pellet was red according to the expression of the Cherry protein. The supernatant was discarded and 100 μl of water was added to resuspend the bacteria. 100 μl of “cracking” buffer (100 mM DTT, 2% SDS, 80 mM Tris-HCl, pH 6.8, 0.006% bromophenol blue, 15% glycerol) was also added to lyse the bacteria. The non-induced samples were treated with the same protocol except that only 60 μl of water and 60 μl of cracking buffer were used (according to the optical density of the samples).
5) The samples were heated at 70° C.-100° C. (10 min.) to resuspend all proteins and to denature the proteins.
6) 10 μl of each sample was loaded on 12% SDS-PAGE gel and migrated during 2 hours at 100 Volt.
7) After migration, the proteins were colored with Coomassie-blue staining.

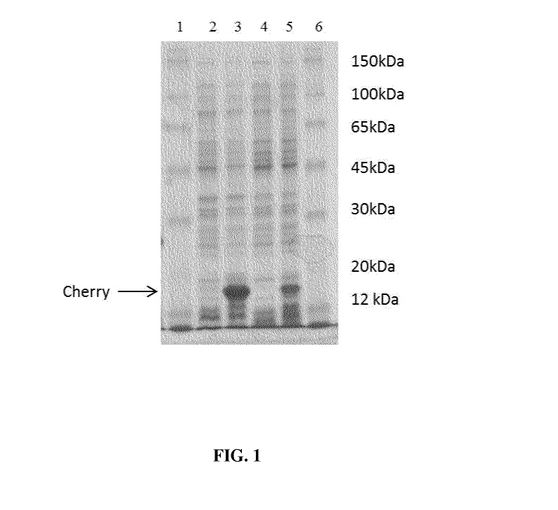

As shown on FIG. 1, both strains were able to produce the protein of interest (Cherry protein, indicated by the array) but the production is about 5 to 10 times higher using M11(DE3) than using HMS174(DE3). The experiment was performed twice with exactly the same results.

### Example 3

### Protein Expression Using BL21(DE3)

The BL21(DE3) strains deleted of Δxis-ea10 and/or ΔS-C were constructed according to example 1. The deletion ΔS-C was inserted in the chromosome of the bacteria (in lambda DE3 deleted of the int gene and encoding the T7 RNA polymerase) alone or in combination with the Δxis-ea10 deletion. Two BL21(DE3) derivatives were thus constructed: BL21(DE3) ΔS-C and BL21(DE3) Δxis-ea10, ΔS-C. The deletions were confirmed by PCR amplification of the modified region and this region was sequenced. This sequencing step confirmed the presence of the corresponding deletions according to the theoretical sequences. Next, the constructed bacteria were tested for protein expression using two different plasmids encoding the cherry protein (pSCherryl and pSSC-Cherryl, Delphi Genetics) according to example 2. The results showed clearly over-expression of the cherry gene encoding the cherry protein. This protein was easily detected by eyes and on SDS-PAGE analysis.

We thus conclude that these bacteria are able to over-produce proteins without risk to unwanted bacterial lysis due to the lambda DE3 phage because this phage is at least deleted of the int, S, R and Rz genes encoding functions required for phage excision and bacterial lysis.

Since bacterial lysis during protein depends on several parameters including growth conditions, protein overexpressed, . . . we designed a model strain to show that the constructed strains are resistant to lysis due to the specific gene deletions. In lambda phage, it is required to have a constitutive expression of the CI repressor in order to repress lytic development and to maintain lysogenic state of the prophage. A mutant (CI⁸⁵⁷) of this repressor is well known due to its ability to be thermosensitive: at 30° C., the mutated repressor is fully active and it maintains the lysogenic state. However, at higher temperature (37° C. to 42° C.), the CI⁸⁵⁷ is not efficient and it induces the lytic state of lambda phage. Strains carrying this CI⁸⁵⁷ mutation and the deletions according to the invention were constructed: BL21(DE3) ΔS-C corresponding to a BL21 strain comprising P41; BL21(DE3) Δxis-ea10 and ΔS-C corresponding to a BL21 strain comprising P11; BL21(DE3) Δxis-ea10, ΔS-C and ΔQ corresponding to a BL21 strain comprising P13; BL21 (DE3) ΔQ corresponding to a BL21 strain comprising P15 and BL21(DE3) Δxis-ea10 and ΔQ corresponding to a BL21 strain comprising P53. By shifting the temperature to 42° C., lytic state is induced. No lysis was observed for the strains of the invention. In addition, we observed that yield of production of the protein of interest (cherry protein) was improved for the strains of the invention compared to control.

1.-15. (canceled)

16. A genetically modified phage comprising:
an inserted expression system;
an inactivated S, R and/or Q gene; and
an inactivated Int and/or Xis gene.

17. The phage of claim 16, comprising an inactivated Rz gene.

18. The phage of claim 16, comprising an inactivated Int gene, an inactivated Xis gene, and inactivated R, S and Rz genes.

19. The phage of claim 18, further comprising an inactivated Q gene.

20. The phage of claim 16, wherein the inserted expression system is a T7 expression system.

21. The phage of claim 16, wherein the phage is a genetically modified lambda phage, 434 phage, phi80 phage, phi81 phage, HK97 phage or P21 phage.

22. The phage of claim 16, wherein the phage is genetically modified phage of any of phages P11 to P53.

23. A kit comprising:
a phage of claim 16; and
a helper phage.

24. A host cell comprising a phage of claim 16.

25. The host cell of claim 24, wherein the host cell is an enterobacteria.

26. The host cell of claim 24, wherein the host cell is E. coli.

27. The host cell of claim 26, wherein the host cell is E. coli BL21.

28. The host cell of claim 24, further comprising at least one inactivated tonA, galK, araB, araA, lon, ompT, rcsA, hsdR, mrr, endA, and/or recA gene.

29. The host cell of claim 24, further comprising an inserted ccdb gene.

30. A kit comprising:
a host cell of claim 29; and
a plasmid comprising the ccdA gene.

31. A process for preparing a host cell, comprising infecting a host cell with a phage of claim 16.

32. A process for producing a biomolecule of interest, comprising:
cultivating a host cell comprising a phage of claim 16 and a nucleic acid sequence of the biomolecule of interest; and
recovering the biomolecule of interest.


SEQUENCE LISTING (36 sequences)

SEQ ID NO: 1 - DNA, 324 bp, Enterobacteria phage lambda
  source 1..324: /mol_type=“DNA” /organism=“Enterobacteria phage lambda”
atgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataa

SEQ ID NO: 2 - DNA, 477 bp, Enterobacteria phage lambda
  source 1..477: /mol_type=“DNA” /organism=“Enterobacteria phage lambda”
atggtagaaatcaataatcaacgtaaggcgttcctcgatatgctggcgtggtcggagggaactgataacggacgtcagaaaaccagaaatcatggttatgacgtcattgtaggcggagagctattcactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatga

SEQ ID NO: 3 - DNA, 462 bp, Enterobacteria phage lambda
  source 1..462: /mol_type=“DNA” /organism=“Enterobacteria phage lambda”
atgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatag

SEQ ID NO: 4 - DNA, 201 bp, Enterobacteria phage lambda
  source 1..201: /mol_type=“DNA” /organism=“Enterobacteria phage lambda”
atgactacgactattgataaaaatcaatggtgtggacaattcaagcgatgcaatggatgcaagctgcaatcggaatgcatggttaagcctgaagaaatgtttcctgtaatggaagatgggaaatatgtcgataaatgggcaatacgaacgacggcaatgattgccagagaacttggtaaacagaacaacaaagctgcctga

SEQ ID NO: 5 - DNA, 300 bp, Enterobacteria phage lambda
  source 1..300: /mol_type=“DNA” /organism=“Enterobacteria phage lambda”
atggtaaccattgtctggaaagaatccaaaggtacggcaaaaagccgctacaaagctcgcagagcagaacttattgccgagcgacgcagtaatgaagcactggcgcgaaaaattgcgctaaagctctctggttgcgtcagagcagataaagcagcatcactcggaagccttcgctgcaagaaggcagaagaagtcgagcgtaaacagaaccgtatttactacagcaagccacgcagtgaaatgggtgtgacttgtgttggtcgccagaaaattaaattaggcagcaaaccacttatttga

SEQ ID NO: 6 - DNA, 651 bp, Enterobacteria phage lambda
  source 1..651: /mol_type=“DNA” /organism=“Enterobacteria phage lambda”
atgaatacacaactgatgggtgagcgtattcgcgctcgcagaaaagaactcaagattaggcaggctgcccttggcaagatggttggcgtgtctaatgttgctatttcccaatgggagcgatctgaaactgagcccaatggcgaaaacctattggccttagccaaggctttgcagtgctcccctgattacctgttgaaaggagaggatagtctttcaaacattgcctatcacagcaggcatgatccaagaggttcgtatcctctaattagttgggtaagcgcaggatgttggatggaagctgtagagccatatcataggcgtgcaatagataactggtacgacacaacggtagattgttctgaagactctttttggctcgacgttaaaggcgattcaatgactgccccggcaggactgagtattcctgaggggatgattattctcgtcgacccagaagtcgaaccacgtaatggaaagctggtagtcgccaaacttgaaggagaaaacgaggcgacattcaaaaagttagttattgatgccggtagaaaattcctgaaaccactcaatccacaatacccaatgattgaaatcaatgggaactgtaaaatcattggcgttgtcgttgatgccaagctagcaaaccttccttaa

SEQ ID NO: 7 - DNA, 1071 bp, Enterobacteria phage lambda
  source 1..1071: /mol_type=“DNA” /organism=“Enterobacteria phage lambda”
atgggaagaaggcgaagtcatgagcgccgggatttaccccctaacctttatataagaaacaatggatattactgctacagggacccaaggacgggtaaagagtttggattaggcagagacaggcgaatcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatcagaggtaaggagatcaagacttacggctgacgaatacctgaaaatttatcaagcagcagaatcatcaccatgttggctcagacttgcaatggaactggctgttgttaccgggcaacgagttggtgatttatgcgaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataa

SEQ ID NO: 8 - DNA, 219 bp, Enterobacteria phage lambda
  source 1..219: /mol_type=“DNA” /organism=“Enterobacteria phage lambda”
atgtacttgacacttcaggagtggaacgcacgccagcgacgtccaagaagccttgaaacagttcgtcgatgggttcgggaatgcaggatattcccacctccggttaaggatggaagagagtatctgttccacgaatcagcggtaaaggttgacttaaatcgaccagtaacaggtggccttttgaagaggatcagaaatgggaagaaggcgaagtcatga

SEQ ID NO: 9 - DNA, 232 bp, Enterobacteria phage lambda
  source 1..232: /mol_type=“DNA” /organism=“Enterobacteria phage lambda”
tacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaagttggcattataaaaaagcattgcttatcaatttgttgcaacgaacaggtcactatcagtcaaaataaaatcattattt

SEQ ID NO: 10 - DNA, 29865 bp, artificial sequences
  source 1..29865: /mol_type=“DNA” /note=“Genetically modified phage” /organism=“artificial sequences”
gcttttttatactaagttggcattataaaaaagcattgcttatcaatttgttgcaacgaacaggtcactatcagtcaaaataaaatcattatttgatttcaattttgtcccactccctgcctctgtcatcacgatactgtgatgccatggtgtccgacttatgcccgagaagatgttgagcaaacttatcgcttatctgcttctcatagagtcttgcagacaaactgcgcaactcgtgaaaggtaggcggatccagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccccaggctttacactttatgcttccggctcgtataatgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgtcgggatctgatccggatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactccggatccccttcgaaggaaagacctgatgcttttcgtgcgcgcataaaataccttgatactgtgccggatgaaagcggttcgcgacgagtagatgcaattatggtttctccgccaagaatctctttgcatttatcaagtgtttccttcattgatattccgagagcatcaatatgcaatgctgttgggatggcaatttttacgcctgttttgctttgctcgacataaagatatccatctacgatatcagaccacttcatttcgcataaatcaccaactcgttgcccggtaacaacagccagttccattgcaagtctgagccaacatggtgatgattctgctgcttgataaattttcaggtattcgtcagccgtaagtcttgatctccttacctctgattttgctgcgcgagtggcagcgacatggtttgttgttatatggccttcagctattgcctctcggaatgcatcgctcagtgttgatctgattaacttggctgacgccgccttgccctcgtctatgtatccattgagcattgccgcaatttcttttgtggtgatgtcttcaagtggagcatcaggcagacccctccttattgctttaattttgctcatgtaatttatgagtgtcttctgcttgattcctctgctggccaggattttttcgtagcgatcaagccatgaatgtaacgtaacggaattatcactgttgattctcgctgtcagaggcttgtgtttgtgtcctgaaaataactcaatgttggcctgtatagcttcagtgattgcgattcgcctgtctctgcctaatccaaactctttacccgtccttgggtccctgtagcagtaatatccattgtttcttatataaaggttagggggtaaatcccggcgctcatgacttcgccttcttcacactggagggcaaagaagatttccaataatcagaacaagtcggctcctgtttagttacgagcgacattgctccgtgtattcactcgttggaatgaatacacagtgcagtgtttattctgttatttatgccaaaaataaaggccactatcaggcagctttgttgttctgtttaccaagttctctggcaatcattgccgtcgttcgtattgcccatttatcgacatatttcccatcttccattacaggaaacatttcttcaggcttaaccatgcattccgattgcagcttgcatccattgcatcgcttgaattgtccacaccattgatttttatcaatagtcgtagtcatacggatagtcctggtattgttccatcacatcctgaggatgctcttcgaactcttcaaattcttcttccatatatcacctcaaataagtggtttgctgcctaatttaattttctggcgaccaacacaagtcacacccatttcactgcgtggcttgctgtagtaaatacggttctgtttacgctcgacttcttctgccttcttgcagcgaaggcttccgagtgatgctgctttatctgctctgacgcaaccagagagctttagcgcaatttttcgcgccagtgcttcattactgcgtcgctcggcaataagttctgctctgcgagctttgtagcggctttttgccgtacctttggattctttccagacaatggttaccatgatggtctcctttaagtggctttggcgcatgacgcgtcgaggtgcttatcttctcgatcgctgtcttgtagctgcaattcgcgccatccccaaaaccactcaagttctggtctcaacggttaggttgagagtccgtcgatgttaaagagcctgccaatctgttccgtttggcttccagcgtcctgctgatggcttaaatttaagacttcttaatttattggtcaagtgcatttttgaagaaaacttaattttatgggcgtgaatttagtttgtctttgatttttaacgggaaataaaaaaggggcgaaagccccttaaggaaggtttgctagcttggcatcaacgacaacgccaatgattttacagttcccattgatttcaatcattgggtattgtggattgagtggtttcaggaattttctaccggcatcaataactaactttttgaatgtcgcctcgttttctccttcaagtttggcgactaccagctttccattacgtggttcgacttctgggtcgacgagaataatcatcccctcaggaatactcagtcctgccggggcagtcattgaatcgcctttaacgtcgagccaaaaagagtcttcagaacaatctaccgttgtgtcgtaccagttatctattgcacgcctatgatatggctctacagcttccatccaacatcctgcgcttacccaactaattagaggatacgaacctcttggatcatgcctgctgtgataggcaatgtttgaaagactatcctctcctttcaacaggtaatcaggggagcactgcaaagccttggctaaggccaataggttttcgccattgggctcagtttcagatcgctcccattgggaaatagcaacattagacacgccaaccatcttgccaagggcagcctgcctaatcttgagttcttttctgcgagcgcgaatacgctcacccatcagttgtgtattcatagttaagacatcttaaataaacttgacttaagattcctttggtggataatttaagtgttctttaatttcggagcgagtctatgtacaaaaaagatgttattgaccacttcggaacccagcgtgctgttgctaaagcactaggcattagcgatgcagcagtctctcagtggaaagaagttatcccagagaaagacgcctatcgattggaaatcgttacagctggcgccctgaagtatcaagaaagtgcttaccgccaagcggcataagcaaattgctctttaacagttctggcctttcacctctaaccgggtgagcaaacatcagcggcaaatccattgggtgtgccgctataactcaatatcaatataggtaaattaacaaatggcacaagcaagctacagcaagccaacacagcgagaaattgatcgcgctgaaactgatttactcatcaacctgtcaacgcttacccagcgcggtctggcaaagatgattggctgtcatgaatcgaagataagcagaacggactggagatttattgcttcggtcttgtgtgctttcggaatggcatcagacatcagtccgattagcagggcttttaagtatgcgcttgatggactcacaaagaaaaaacgcccggtgtgcaagaccgagcgttctgaacaaatccagatggagttctgaggtcattactggatctatcaacaggagtcattatgacaaatacagcaaaaatactcaacttcggcagaggtaactttgccggacaggagcgtaatgtggcagatctcgatgatggttacgccagactatcaaatatgctgcttgaggcttattcgggcgcagatctgaccaagcgacagtttaaagtgctgcttgccattctgcgtaaaacctatgggtggaataaaccaatggacagaatcaccgattctcaacttagcgagattacaaagttacctgtcaaacggtgcaatgaagccaagttagaactcgtcagaatgaatattatcaagcagcaaggcggcatgtttggaccaaataaaaacatctcagaatggtgtatccctcaaaacgagggaaaatcccctaaaacgagggataaaacatccctcaaattgggggattgctatccctcaaaacagggggacacaaaagacactattacaaaagaaaaaagaaaagattattcgtcagagaattctggcgaatcctctgaccagccagaaaacgacctttctgtggtgaaaccggatgctgcaattcagagcggcagcaagtgggggacagcagaagacctgaccgccgcagagtggatgtttgacatggtgaagactatcgcaccatcagccagaaaaccgaattttgctgggtgggctaacgatatccgcctgatgcgtgaacgtgacggacgtaaccaccgcgacatgtgtgtgctgttccgctgggcatgccaggacaacttctggtccggtaacgtgctgagcccggccaaactccgcgataagtggacccaactcgaaatcaaccgtaacaagcaacaggcaggcgtgacagccagcaaaccaaaactcgacctgacaaacacagactggatttacggggtggatctatgaaaaacatcgccgcacagatggttaactttgaccgtgagcagatgcgtcggatcgccaacaacatgccggaacagtacgacgaaaagccgcaggtacagcaggtagcgcagatcatcaacggtgtgttcagccagttactggcaactttcccggcgagcctggctaaccgtgaccagaacgaagtgaacgaaatccgtcgccagtgggttctggcttttcgggaaaacgggatcaccacgatggaacaggttaacgcaggaatgcgcgtagcccgtcggcagaatcgaccatttctgccatcacccgggcagtttgttgcatggtgccgggaagaagcatccgttaccgccggactgccaaacgtcagcgagctggttgatatggtttacgagtattgccggaagcgaggcctgtatccggatgcggagtcttatccgtggaaatcaaacgcgcactactggctggttaccaacctgtatcagaacatgcgggccaatgcgcttactgatgcggaattacgccgtaaggccgcagatgagcttgtccatatgactgcgagaattaaccgtggtgaggcgatccctgaaccagtaaaacaacttcctgtcatgggcggtagacctctaaatcgtgcacaggctctggcgaagatcgcagaaatcaaagctaagttcggactgaaaggagcaagtgtatgacgggcaaagaggcaattattcattacctggggacgcataatagcttctgtgcgccggacgttgccgcgctaacaggcgcaacagtaaccagcataaatcaggccgcggctaaaatggcacgggcaggtcttctggttatcgaaggtaaggtctggcgaacggtgtattaccggtttgctaccagggaagaacgggaaggaaagatgagcacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcatatcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattccatgcttgaacccgcctatgcgcgggttttcttttgtgcgcttgcaggccagcttgggatcagcagcctgacggatgcggtgtccggcgacagcctgactgcccaggaggcactcgcgacgctggcattatccggtgatgatgacggaccacgacaggcccgcagttatcaggtcatgaacggcatcgccgtgctgccggtgtccggcacgctggtcagccggacgcgggcgctgcagccgtactcggggatgaccggttacaacggcattatcgcccgtctgcaacaggctgccagcgatccgatggtggacggcattctgctcgatatggacacgcccggcgggatggtggcgggggcatttgactgcgctgacatcatcgcccgtgtgcgtgacataaaaccggtatgggcgcttgccaacgacatgaactgcagtgcaggtcagttgcttgccagtgccgcctcccggcgtctggtcacgcagaccgcccggacaggctccatcggcgtcatgatggctcacagtaattacggtgctgcgctggagaaacagggtgtggaaatcacgctgatttacagcggcagccataaggtggatggcaacccctacagccatcttccggatgacgtccgggagacactgcagtcccggatggacgcaacccgccagatgtttgcgcagaaggtgtcggcatataccggcctgtccgtgcaggttgtgctggataccgaggctgcagtgtacagcggtcaggaggccattgatgccggactggctgatgaacttgttaacagcaccgatgcgatcaccgtcatgcgtgatgcactggatgcacgtaaatcccgtctctcaggagggcgaatgaccaaagagactcaatcaacaactgtttcagccactgcttcgcaggctgacgttactgacgtggtgccagcgacggagggcgagaacgccagcgcggcgcagccggacgtgaacgcgcagatcaccgcagcggttgcggcagaaaacagccgcattatggggatactcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagtgaatccgcagatgaccctgcgtcgcctgccggatgaagatccgcagaatctggcggacccggcttaccgccgccgtcgcatcatcatgcagaacatgcgtgacgaagagctggccattgctcaggtcgaagagatgcaggcagtttctgccgtgcttaagggcaaatacaccatgaccggtgaagccttcgatccggttgaggtggatatgggccgcagtgaggagaataacatcacgcagtccggcggcacggagtggagcaagcgtgacaagtccacgtatgacccgaccgacgatatcgaagcctacgcgctgaacgccagcggtgtggtgaatatcatcgtgttcgatccgaaaggctgggcgctgttccgttccttcaaagccgtcaaggagaagctggatacccgtcgtggctctaattccgagctggagacagcggtgaaagacctgggcaaagcggtgtcctataaggggatgtatggcgatgtggccatcgtcgtgtattccggacagtacgtggaaaacggcgtcaaaaagaacttcctgccggacaacacgatggtgctggggaacactcaggcacgcggtctgcgcacctatggctgcattcaggatgcggacgcacagcgcgaaggcattaacgcctctgcccgttacccgaaaaactgggtgaccaccggcgatccggcgcgtgagttcaccatgattcagtcagcaccgctgatgctgctggctgaccctgatgagttcgtgtccgtacaactggcgtaatcatggcccttcggggccattgtttctctgtggaggagtccatgacgaaagatgaactgattgcccgtctccgctcgctgggtgaacaactgaaccgtgatgtcagcctgacggggacgaaagaagaactggcgctccgtgtggcagagctgaaagaggagcttgatgacacggatgaaactgccggtcaggacacccctctcagccgggaaaatgtgctgaccggacatgaaaatgaggtgggatcagcgcagccggataccgtgattctggatacgtctgaactggtcacggtcgtggcactggtgaagctgcatactgatgcacttcacgccacgcgggatgaacctgtggcatttgtgctgccgggaacggcgtttcgtgtctctgccggtgtggcagccgaaatgacagagcgcggcctggccagaatgcaataacgggaggcgctgtggctgatttcgataacctgttcgatgctgccattgcccgcgccgatgaaacgatacgcgggtacatgggaacgtcagccaccattacatccggtgagcagtcaggtgcggtgatacgtggtgtttttgatgaccctgaaaatatcagctatgccggacagggcgtgcgcgttgaaggctccagcccgtccctgtttgtccggactgatgaggtgcggcagctgcggcgtggagacacgctgaccatcggtgaggaaaatttctgggtagatcgggtttcgccggatgatggcggaagttgtcatctctggcttggacggggcgtaccgcctgccgttaaccgtcgccgctgaaagggggatgtatggccataaaaggtcttgagcaggccgttgaaaacctcagccgtatcagcaaaacggcggtgcctggtgccgccgcaatggccattaaccgcgttgcttcatccgcgatatcgcagtcggcgtcacaggttgcccgtgagacaaaggtacgccggaaactggtaaaggaaagggccaggctgaaaagggccacggtcaaaaatccgcaggccagaatcaaagttaaccggggggatttgcccgtaatcaagctgggtaatgcgcgggttgtcctttcgcgccgcaggcgtcgtaaaaaggggcagcgttcatccctgaaaggtggcggcagcgtgcttgtggtgggtaaccgtcgtattcccggcgcgtttattcagcaactgaaaaatggccggtggcatgtcatgcagcgtgtggctgggaaaaaccgttaccccattgatgtggtgaaaatcccgatggcggtgccgctgaccacggcgtttaaacaaaatattgagcggatacggcgtgaacgtcttccgaaagagctgggctatgcgctgcagcatcaactgaggatggtaataaagcgatgaaacatactgaactccgtgcagccgtactggatgcactggagaagcatgacaccggggcgacgttttttgatggtcgccccgctgtttttgatgaggcggattttccggcagttgccgtttatctcaccggcgctgaatacacgggcgaagagctggacagcgatacctggcaggcggagctgcatatcgaagttttcctgcctgctcaggtgccggattcagagctggatgcgtggatggagtcccggatttatccggtgatgagcgatatcccggcactgtcagatttgatcaccagtatggtggccagcggctatgactaccggcgcgacgatgatgcgggcttgtggagttcagccgatctgacttatgtcattacctatgaaatgtgaggacgctatgcctgtaccaaatcctacaatgccggtgaaaggtgccgggaccaccctgtgggtttataaggggagcggtgacccttacgcgaatccgctttcagacgttgactggtcgcgtctggcaaaagttaaagacctgacgcccggcgaactgaccgctgagtcctatgacgacagctatctcgatgatgaagatgcagactggactgcgaccgggcaggggcagaaatctgccggagataccagcttcacgctggcgtggatgcccggagagcaggggcagcaggcgctgctggcgtggtttaatgaaggcgatacccgtgcctataaaatccgcttcccgaacggcacggtcgatgtgttccgtggctgggtcagcagtatcggtaaggcggtgacggcgaaggaagtgatcacccgcacggtgaaagtcaccaatgtgggacgtccgtcgatggcagaagatcgcagcacggtaacagcggcaaccggcatgaccgtgacgcctgccagcacctcggtggtgaaagggcagagcaccacgctgaccgtggccttccagccggagggcgtaaccgacaagagctttcgtgcggtgtctgcggataaaacaaaagccaccgtgtcggtcagtggtatgaccatcaccgtgaacggcgttgctgcaggcaaggtcaacattccggttgtatccggtaatggtgagtttgctgcggttgcagaaattaccgtcaccgccagttaatccggagagtcagcgatgttcctgaaaaccgaatcatttgaacataacggtgtgaccgtcacgctttctgaactgtcagccctgcagcgcattgagcatctcgccctgatgaaacggcaggcagaacaggcggagtcagacagcaaccggaagtttactgtggaagacgccatcagaaccggcgcgtttctggtggcgatgtccctgtggcataaccatccgcagaagacgcagatgccgtccatgaatgaagccgttaaacagattgagcaggaagtgcttaccacctggcccacggaggcaatttctcatgctgaaaacgtggtgtaccggctgtctggtatgtatgagtttgtggtgaataatgcccctgaacagacagaggacgccgggcccgcagagcctgtttctgcgggaaagtgttcgacggtgagctgagttttgccctgaaactggcgcgtgagatggggcgacccgactggcgtgccatgcttgccgggatgtcatccacggagtatgccgactggcaccgcttttacagtacccattattttcatgatgttctgctggatatgcacttttccgggctgacgtacaccgtgctcagcctgtttttcagcgatccggatatgcatccgctggatttcagtctgctgaaccggcgcgaggctgacgaagagcctgaagatgatgtgctgatgcagaaagcggcagggcttgccggaggtgtccgctttggcccggacgggaatgaagttatccccgcttccccggatgtggcggacatgacggaggatgacgtaatgctgatgacagtatcagaagggatcgcaggaggagtccggtatggctgaaccggtaggcgatctggtcgttgatttgagtctggatgcggccagatttgacgagcagatggccagagtcaggcgtcatttttctggtacggaaagtgatgcgaaaaaaacagcggcagtcgttgaacagtcgctgagccgacaggcgctggctgcacagaaagcggggatttccgtcgggcagtataaagccgccatgcgtatgctgcctgcacagttcaccgacgtggccacgcagcttgcaggcgggcaaagtccgtggctgatcctgctgcaacagggggggcaggtgaaggactccttcggcgggatgatccccatgttcagggggcttgccggtgcgatcaccctgccgatggtgggggccacctcgctggcggtggcgaccggtgcgctggcgtatgcctggtatcagggcaactcaaccctgtccgatttcaacaaaacgctggtcctttccggcaatcaggcgggactgacggcagatcgtatgctggtcctgtccagagccgggcaggcggcagggctgacgtttaaccagaccagcgagtcactcagcgcactggttaaggcgggggtaagcggtgaggctcagattgcgtccatcagccagagtgtggcgcgtttctcctctgcatccggcgtggaggtggacaaggtcgctgaagccttcgggaagctgaccacagacccgacgtcggggctgacggcgatggctcgccagttccataacgtgtcggcggagcagattgcgtatgttgctcagttgcagcgttccggcgatgaagccggggcattgcaggcggcgaacgaggccgcaacgaaagggtttgatgaccagacccgccgcctgaaagagaacatgggcacgctggagacctgggcagacaggactgcgcgggcattcaaatccatgtgggatgcggtgctggatattggtcgtcctgataccgcgcaggagatgctgattaaggcagaggctgcgtataagaaagcagacgacatctggaatctgcgcaaggatgattattttgttaacgatgaagcgcgggcgcgttactgggatgatcgtgaaaaggcccgtcttgcgcttgaagccgcccgaaagaaggctgagcagcagactcaacaggacaaaaatgcgcagcagcagagcgataccgaagcgtcacggctgaaatataccgaagaggcgcagaaggcttacgaacggctgcagacgccgctggagaaatataccgcccgtcaggaagaactgaacaaggcactgaaagacgggaaaatcctgcaggcggattacaacacgctgatggcggcggcgaaaaaggattatgaagcgacgctgaaaaagccgaaacagtccagcgtgaaggtgtctgcgggcgatcgtcaggaagacagtgctcatgctgccctgctgacgcttcaggcagaactccggacgctggagaagcatgccggagcaaatgagaaaatcagccagcagcgccgggatttgtggaaggcggagagtcagttcgcggtactggaggaggcggcgcaacgtcgccagctgtctgcacaggagaaatccctgctggcgcataaagatgagacgctggagtacaaacgccagctggctgcacttggcgacaaggttacgtatcaggagcgcctgaacgcgctggcgcagcaggcggataaattcgcacagcagcaacgggcaaaacgggccgccattgatgcgaaaagccgggggctgactgaccggcaggcagaacgggaagccacggaacagcgcctgaaggaacagtatggcgataatccgctggcgctgaataacgtcatgtcagagcagaaaaagacctgggcggctgaagaccagcttcgcgggaactggatggcaggcctgaagtccggctggagtgagtgggaagagagcgccacggacagtatgtcgcaggtaaaaagtgcagccacgcagacctttgatggtattgcacagaatatggcggcgatgctgaccggcagtgagcagaactggcgcagcttcacccgttccgtgctgtccatgatgacagaaattctgcttaagcaggcaatggtggggattgtcgggagtatcggcagcgccattggcggggctgttggtggcggcgcatccgcgtcaggcggtacagccattcaggccgctgcggcgaaattccattttgcaaccggaggatttacgggaaccggcggcaaatatgagccagcggggattgttcaccgtggtgagtttgtcttcacgaaggaggcaaccagccggattggcgtggggaatctttaccggctgatgcgcggctatgccaccggcggttatgtcggtacaccgggcagcatggcagacagccggtcgcaggcgtccgggacgtttgagcagaataaccatgtggtgattaacaacgacggcacgaacgggcagataggtccggctgctctgaaggcggtgtatgacatggcccgcaagggtgcccgtgatgaaattcagacacagatgcgtgatggtggcctgttctccggaggtggacgatgaagaccttccgctggaaagtgaaacccggtatggatgtggcttcggtcccttctgtaagaaaggtgcgctttggtgatggctattctcagcgagcgcctgccgggctgaatgccaacctgaaaacgtacagcgtgacgctttctgtcccccgtgaggaggccacggtactggagtcgtttctggaagagcacgggggctggaaatcctttctgtggacgccgccttatgagtggcggcagataaaggtgacctgcgcaaaatggtcgtcgcgggtcagtatgctgcgtgttgagttcagcgcagagtttgaacaggtggtgaactgatgcaggatatccggcaggaaacactgaatgaatgcacccgtgcggagcagtcggccagcgtggtgctctgggaaatcgacctgacagaggtcggtggagaacgttattttttctgtaatgagcagaacgaaaaaggtgagccggtcacctggcaggggcgacagtatcagccgtatcccattcaggggagcggttttgaactgaatggcaaaggcaccagtacgcgccccacgctgacggtttctaacctgtacggtatggtcaccgggatggcggaagatatgcagagtctggtcggcggaacggtggtccggcgtaaggtttacgcccgttttctggatgcggtgaacttcgtcaacggaaacagttacgccgatccggagcaggaggtgatcagccgctggcgcattgagcagtgcagcgaactgagcgcggtgagtgcctcctttgtactgtccacgccgacggaaacggatggcgctgtttttccgggacgtatcatgctggccaacacctgcacctggacctatcgcggtgacgagtgcggttatagcggtccggctgtcgcggatgaatatgaccagccaacgtccgatatcacgaaggataaatgcagcaaatgcctgagcggttgtaagttccgcaataacgtcggcaactttggcggcttcctttccattaacaaactttcgcagtaaatcccatgacacagacagaatcagcgattctggcgcacgcccggcgatgtgcgccagcggagtcgtgcggcttcgtggtaagcacgccggagggggaaagatatttcccctgcgtgaatatctccggtgagccggaggcgtatttccgtatgtcgccggaagactggctgcaggcagaaatgcagggtgagattgtggcgctggtccacagccaccccggtggtctgccctggctgagtgaggccgaccggcggctgcaggtgcagagtgatttgccgtggtggctggtctgccgggggacgattcataagttccgctgtgtgccgcatctcaccgggcggcgctttgagcacggtgtgacggactgttacacactgttccgggatgcttatcatctggcggggattgagatgccggactttcatcgtgaggatgactggtggcgtaacggccagaatctctatctggataatctggaggcgacggggctgtatcaggtgccgttgtcagcggcacagccgggcgatgtgctgctgtgctgttttggttcatcagtgccgaatcacgccgcaatttactgcggcgacggcgagctgctgcaccatattcctgaacaactgagcaaacgagagaggtacaccgacaaatggcagcgacgcacacactccctctggcgtcaccgggcatggcgcgcatctgcctttacggggatttacaacgatttggtcgccgcatcgaccttcgtgtgaaaacgggggctgaagccatccgggcactggccacacagctcccggcgtttcgtcagaaactgagcgacggctggtatcaggtacggattgccgggcgggacgtcagcacgtccgggttaacggcgcagttacatgagactctgcctgatggcgctgtaattcatattgttcccagagtcgccggggccaagtcaggtggcgtattccagattgtcctgggggctgccgccattgccggatcattctttaccgccggagccacccttgcagcatggggggcagccattggggccggtggtatgaccggcatcctgttttctctcggtgccagtatggtgctcggtggtgtggcgcagatgctggcaccgaaagccagaactccccgtatacagacaacggataacggtaagcagaacacctatttctcctcactggataacatggttgcccagggcaatgttctgcctgttctgtacggggaaatgcgcgtggggtcacgcgtggtttctcaggagatcagcacggcagacgaaggggacggtggtcaggttgtggtgattggtcgctgatgcaaaatgttttatgtgaaaccgcctgcgggcggttttgtcatttatggagcgtgaggaatgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacataccgcttcacgcaactggcgctggggaactacaggctgacagtccgggcggtaaatgcgtgggggcagcagggcgatccggcgtcggtatcgttccggattgccgcaccggcagcaccgtcgaggattgagctgacgccgggctattttcagataaccgccacgccgcatcttgccgtttatgacccgacggtacagtttgagttctggttctcggaaaagcagattgcggatatcagacaggttgaaaccagcacgcgttatcttggtacggcgctgtactggatagccgccagtatcaatatcaaaccgggccatgattattacttttatatccgcagtgtgaacaccgttggcaaatcggcattcgtggaggccgtcggtcgggcgagcgatgatgcggaaggttacctggattttttcaaaggcaagataaccgaatcccatctcggcaaggagctgctggaaaaagtcgagctgacggaggataacgccagcagactggaggagttttcgaaagagtggaaggatgccagtgataagtggaatgccatgtgggctgtcaaaattgagcagaccaaagacggcaaacattatgtcgcgggtattggcctcagcatggaggacacggaggaaggcaaactgagccagtttctggttgccgccaatcgtatcgcatttattgacccggcaaacgggaatgaaacgccgatgtttgtggcgcagggcaaccagatattcatgaacgacgtgttcctgaagcgcctgacggcccccaccattaccagcggcggcaatcctccggccttttccctgacaccggacggaaagctgaccgctaaaaatgcggatatcagtggcagtgtgaatgcgaactccgggacgctcagtaatgtgacgatagctgaaaactgtacgataaacggtacgctgagggcggaaaaaatcgtcggggacattgtaaaggcggcgagcgcggcttttccgcgccagcgtgaaagcagtgtggactggccgtcaggtacccgtactgtcaccgtgaccgatgaccatccttttgatcgccagatagtggtgcttccgctgacgtttcgcggaagtaagcgtactgtcagcggcaggacaacgtattcgatgtgttatctgaaagtactgatgaacggtgcggtgatttatgatggcgcggcgaacgaggcggtacaggtgttctcccgtattgttgacatgccagcgggtcggggaaacgtgatcctgacgttcacgcttacgtccacacggcattcggcagatattccgccgtatacgtttgccagcgatgtgcaggttatggtgattaagaaacaggcgctgggcatcagcgtggtctgagtgtgttacagaggttcgtccgggaacgggcgttttattataaaacagtgagaggtgaacgatgcgtaatgtgtgtattgccgttgctgtctttgccgcacttgcggtgacagtcactccggcccgtgcggaaggtggacatggtacgtttacggtgggctattttcaagtgaaaccgggtacattgccgtcgttgtcgggcggggataccggtgtgagtcatctgaaagggattaacgtgaagtaccgttatgagctgacggacagtgtgggggtgatggcttccctggggttcgccgcgtcgaaaaagagcagcacagtgatgaccggggaggatacgtttcactatgagagcctgcgtggacgttatgtgagcgtgatggccggaccggttttacaaatcagtaagcaggtcagtgcgtacgccatggccggagtggctcacagtcggtggtccggcagtacaatggattaccgtaagacggaaatcactcccgggtatatgaaagagacgaccactgccagggacgaaagtgcaatgcggcatacctcagtggcgtggagtgcaggtatacagattaatccggcagcgtccgtcgttgttgatattgcttatgaaggctccggcagtggcgactggcgtactgacggattcatcgttggggtcggttataaattctgattagccaggtaacacagtgttatgacagcccgccggaaccggtgggcttttttgtggggtgaatatggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcagaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgaaacgaacagaaaagcccactggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattctaagcggagatcgcctagtgattttaaactattgctggcagcattcttgagtccaatataaaagtattgtgtaccttttgctgggtcaggttgttctttaggaggagtaaaaggatcaaatgcactaaacgaaactgaaacaagcgatcgaaaatatccctttgggattcttgactcgataagtctattattttcagagaaaaaatattcattgttttctgggttggtgattgcaccaatcattccattcaaaattgttgttttaccacacccattccgcccgataaaagcatgaatgttcgtgctgggcatagaattaaccgtcacctcaaaaggtatagttaaatcactgaatccgggagcactttttctattaaatgaaaagtggaaatctgacaattctggcaaaccatttaacacacgtgcgaactgtccatgaatttctgaaagagttacccctctaagtaatgaggtgttaaggacgctttcattttcaatgtcggctaatcgatttggccatactactaaatcctgaatagctttaagaaggttatgtttaaaaccatcgcttaatttgctgagattaacatagtagtcaatgctttcacctaaggaaaaaaacatttcagggagttgactgaattttttatctattaatgaataagtgcttacttcttctttttgacctacaaaaccaattttaacatttccgatatcgcatttttcaccatgctcatcaaagacagtaagataaaacattgtaacaaaggaatagtcattccaaccatctgctcgtaggaatgccttatttttttctactgcaggaatatacccgcctctttcaataacactaaactccaacatatagtaacccttaattttattaaaataaccgcaatttatttggcggcaacacaggatctctcttttaagttactctctattacatacgttttccatctaaaaattagtagtattgaacttaacggggcatcgtattgtagttttccatatttagctttctgcttccttttggataacccactgttattcatgttgcatggtgcactgtttataccaacgatatagtctattaatgcatatatagtatcgccgaacgattagctcttcaggcttctgaagaagcgtttcaagtactaataagccgatagatagccacggacttcgtagccatttttcataagtgttaacttccgctcctcgctcataacagacattcactacagttatggcggaaaggtatgcatgctgggtgtggggaagtcgtgaaagaaaagaagtcagctgcgtcgtttgacatcactgctatcttcttactggttatgcaggtcgtagtgggtggcacacaaagctttgcactggattgcgaggctttgtgcttctctggagtgcgacaggtttgatgacaaaaaattagcgcaagaagacaaaaatcaccttgcgctaatgctctgttacaggtcactaataccatctaagtagttgattcatagtgactgcatatgttgtgttttacagtattatgtagtctgttttttatgcaaaatctaatttaatatattgatatttatatcattttacgtttctcgttca

SEQ ID NO: 11 - DNA, 2244 bp, Escherichia coli
  source 1..2244: /mol_type=“DNA” /organism=“Escherichia coli”
atggcgcgttccaaaactgctcagccaaaacactcactgcgtaaaatcgcagttgtagtagccacagcggttagcggcatgtctgtttatgcacaggcagcggttgaaccgaaagaagacactatcaccgttaccgctgcacctgcgccgcaagaaagcgcatgggggcctgctgcaactattgcggcgcgacagtctgctaccggcactaaaaccgatacgccgattcaaaaagtgccacagtctatttctgttgtgaccgccgaagagatggcgctgcatcagccgaagtcggtaaaagaagcgcttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 12 - DNA, 1149 bp, Escherichia coli
  source 1..1149: /mol_type=“DNA” /organism=“Escherichia coli”
atgagtctgaaagaaaaaacacaatctctgtttgccaacgcatttggctaccctgccactcacaccattcaggcgcctggccgcgtgaatttgattggtgaacacaccgactacaacgacggtttcgttctgccctgcgcgattgattatcaaaccgtgatcagttgtgcaccacgcgatgaccgtaaagttcgcgtgatggcagccgattatgaaaatcagctcgacgagttttccctcgatgcgcccattgtcgcacatgaaaactatcaatgggctaactacgttcgtggcgtggtgaaacatctgcaactgcgtaacaacagcttcggcggcgtggacatggtgatcagcggcaatgtgccgcagggtgccgggttaagttcttccgcttcactggaagtcgcggtcggaaccgtattgcagcagctttatcatctgccgctggacggcgcacaaatcgcgcttaacggtcaggaagcagaaaaccagtttgtaggctgtaactgcgggatcatggatcagctaatttccgcgctcggcaagaaagatcatgccttgctgatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcagccagccctgcgtgatgtcaccattgaagagttcaacgctgttgcgcatgaactggacccgatcgtggcaaaacgcgtgcgtcatatactgactgaaaacgcccgcaccgttgaagctgccagcgcgctggagcaaggcgacctgaaacgtatgggcgagttgatggcggagtctcatgcctctatgcgcgatgatttcgaaatcaccgtgccgcaaattgacactctggtagaaatcgtcaaagctgtgattggcgacaaaggtggcgtacgcatgaccggcggcggatttggcggctgtatcgtcgcgctgatcccggaagagctggtgcctgccgtacagcaagctgtcgctgaacaatatgaagcaaaaacaggtattaaagagactttttacgtttgtaaaccatcacaaggagcaggacagtgctga

SEQ ID NO: 13 - DNA, 1701 bp, Escherichia coli
  source 1..1701: /mol_type=“DNA” /organism=“Escherichia coli”
atggcgattgcaattggcctcgattttggcagtgattctgtgcgagctttggcggtggactgcgctaccggtgaagagatcgccaccagcgtagagtggtatccccgttggcagaaagggcaattttgtgatgccccgaataaccagttccgtcatcatccgcgtgactacattgagtcaatggaagcggcactgaaaaccgtgcttgcagagcttagcgtcgaacagcgcgcagctgtggtcgggattggcgttgacagtaccggctcgacgcccgcaccgattgatgccgacggaaacgtgctggcgctgcgcccggagtttgccgaaaacccgaacgcgatgttcgtattgtggaaagaccacactgcggttgaagaagcggaagagattacccgtttgtgccacgcgccgggcaacgttgactactcccgctacattggtggtatttattccagcgaatggttctgggcaaaaatcctgcatgtgactcgccaggacagcgccgtggcgcaatctgccgcatcgtggattgagctgtgcgactgggtgccagctctgctttccggtaccacccgcccgcaggatattcgtcgcggacgttgcagcgccgggcataaatctctgtggcacgaaagctggggcggcctgccgccagccagtttctttgatgagctggacccgatcctcaatcgccatttgccttccccgctgttcactgacacttggactgccgatattccggtgggcaccttatgcccggaatgggcgcagcgtctcggcctgcctgaaagcgtggtgatttccggcggcgcgtttgactgccatatgggcgcagttggcgcaggcgcacagcctaacgcactggtaaaagttatcggtacttccacctgcgacattctgattgccgacaaacagagcgttggcgagcgggcagttaaaggtatttgcggtcaggttgatggcagcgtggtgcctggatttatcggtctggaagcaggccaatcggcgtttggtgatatctacgcctggtttggtcgcgtactcggctggccgctggaacagcttgccgcccagcatccggaactgaaaacgcaaatcaacgccagccagaaacaactgcttccggcgctgaccgaagcatgggccaaaaatccgtctctggatcacctgccggtggtgctcgactggtttaacggccgccgcacaccgaacgctaaccaacgcctgaaaggggtgattaccgatcttaacctcgctaccgacgctccgctgctgttcggcggtttgattgctgccaccgcctttggcgcacgcgcaatcatggagtgctttaccgatcaggggatcgccgttaataacgtgatggcactgggcggcatcgcgcggaaaaaccaggtcattatgcaggcctgctgcgacgtgctgaatcgcccgctgcaaattgttgcctctgaccagtgctgtgcgctcggtgcggcgatttttgctgccgtcgccgcgaaagtgcacgcagacatcccatcagctcagcaaaaaatggccagtgcggtagagaaaaccctgcaaccgtgcagcgagcaggcacaacgctttgaacagctttatcgccgctatcagcaatgggcgatgagcgccgaacaacactatcttccaacttccgccccggcacaggctgcccaggccgttgcgactctataa

SEQ ID NO: 14 - DNA, 1503 bp, Escherichia coli
  source 1..1503: /mol_type=“DNA” /organism=“Escherichia coli”
atgacgatttttgataattatgaagtgtggtttgtcattggcagccagcatctgtatggcccggaaaccctgcgtcaggtcacccaacatgccgagcacgtcgttaatgcgctgaatacggaagcgaaactgccctgcaaactggtgttgaaaccgctgggcaccacgccggatgaaatcaccgctatttgccgcgacgcgaattacgacgatcgttgcgctggtctggtggtgtggctgcacaccttctccccggccaaaatgtggatcaacggcctgaccatgctcaacaaaccgttgctgcaattccacacccagttcaacgcggcgctgccgtgggacagtatcgatatggactttatgaacctgaaccagactgcacatggcggtcgcgagttcggcttcattggcgcgcgtatgcgtcagcaacatgccgtggttaccggtcactggcaggataaacaagcccatgagcgtatcggctcctggatgcgtcaggcggtctctaaacaggatacccgtcatctgaaagtctgccgatttggcgataacatgcgtgaagtggcggtcaccgatggcgataaagttgccgcacagatcaagttcggtttctccgtcaatacctgggcggttggcgatctggtgcaggtggtgaactccatcagcgacggcgatgttaacgcgctggtcgatgagtacgaaagctgctacaccatgacgcctgccacacaaatccacggcaaaaaacgacagaacgtgctggaagcggcgcgtattgagctggggatgaagcgtttcctggaacaaggtggcttccacgcgttcaccaccacctttgaagatttgcacggtctgaaacagcttcctggtctggccgtacagcgtctgatgcagcagggttacggctttgcgggcgaaggcgactggaaaactgccgccctgcttcgcatcatgaaggtgatgtcaaccggtctgcagggcggcacctcctttatggaggactacacctatcacttcgagaaaggtaatgacctggtgctcggctcccatatgctggaagtctgcccgtcgatcgccgcagaagagaaaccgatcctcgacgttcagcatctcggtattggtggtaaggacgatcctgcccgcctgatcttcaatacccaaaccggcccagcgattgtcgccagcttgattgatctcggcgatcgttaccgtctactggttaactgcatcgacacggtgaaaacaccgcactccctgccgaaactgccggtggcgaatgcgctgtggaaagcgcaaccggatctgccaactgcttccgaagcgtggatcctcgctggtggcgcgcaccataccgtcttcagccatgcactgaacctcaacgatatgcgccaattcgccgagatgcacgacattgaaatcacggtgattgataacgacacacgcctgccagcgtttaaagacgcgctgcgctggaacgaagtgtattacgggtttcgtcgctaa

SEQ ID NO: 15 - DNA, 2355 bp, Escherichia coli
  source 1..2355: /mol_type=“DNA” /organism=“Escherichia coli”
atgaatcctgagcgttctgaacgcattgaaatccccgtattgccgctgcgcgatgtggtggtttatccgcacatggtcatccccttatttgtcgggcgggaaaaatctatccgttgtctggaagcggcgatggaccatgataaaaaaattatgctggtcgcgcagaaagaagcttcaacggatgagccgggtgtaaacgatcttttcaccgtcgggaccgtggcctctatattgcagatgctgaaactgcctgacggcaccgtcaaagtgctggtcgaggggttacagcgcgcgcgtatttctgcgctctctgacaatggcgaacacttttctgcgaaggcggagtatctggagtcgccgaccattgatgagcgggaacaggaagtgctggtgcgtactgcaatcagccagttcgaaggctacatcaagctgaacaaaaaaatcccaccagaagtgctgacgtcgctgaatagcatcgacgatccggcgcgtctggcggataccattgctgcacatatgccgctgaaactggctgacaaacagtctgttctggagatgtccgacgttaacgaacgtctggaatatctgatggcaatgatggaatcggaaatcgatctgctgcaggttgagaaacgcattcgcaaccgcgttaaaaagcagatggagaaatcccagcgtgagtactatctgaacgagcaaatgaaagctattcagaaagaactcggtgaaatggacgacgcgccggacgaaaacgaagccctgaagcgcaaaatcgacgcggcgaagatgccgaaagaggcaaaagagaaagcggaagcagagttgcagaagctgaaaatgatgtctccgatgtcggcagaagcgaccgtagtgcgtggttatatcgactggatggtacaggtgccgtggaatgcgcgtagcaaggtcaaaaaagacctgcgtcaggcgcaggaaatccttgataccgaccattatggtctggagcgcgtgaaagatcgaatccttgagtatcttgcggttcaaagccgtgtcaacaaaatcaagggaccgatcctctgcctggtagggccgccgggggtaggtaaaacctctcttggtcagtccattgccaaagccaccgggcgtaaatatgtccgtatggcgctgggcggcgtgcgtgatgaagcggaaatccgtggtcaccgccgtacttacatcggttctatgccgggtaaactgatccagaaaatggcgaaagtgggcgtgaaaaacccgctgttcctgctcgatgagatcgacaaaatgtcttctgacatgcgtggcgatccggcctctgcactgcttgaagtgctggatccagagcagaacgtagcgttcagcgaccactacctggaagtggattacgatctcagcgacgtgatgtttgtcgcgacgtcgaactccatgaacattccggcaccgctgctggatcgtatggaagtgattcgcctctccggttataccgaagatgaaaaactgaacatcgccaaacgtcacctgctgccgaagcagattgaacgtaatgcactgaaaaaaggtgagctgaccgtcgacgatagcgccattatcggcattattcgttactacacccgtgaggcgggcgtgcgtggtctggagcgtgaaatctccaaactgtgtcgcaaagcggttaagcagttactgctcgataagtcattaaaacatatcgaaattaacggcgataacctgcatgactatctcggtgttcagcgtttcgactatggtcgcgcggataacgaaaaccgtgtcggtcaggtaaccggtctggcgtggacggaagtgggcggtgacttgctgaccattgaaaccgcatgtgttccgggtaaaggcaaactgacctataccggttcgctcggcgaagtgatgcaggagtccattcaggcggcgttaacggtggttcgtgcgcgtgcggaaaaactggggatcaaccctgatttttacgaaaaacgtgacatccacgtccacgtaccggaaggtgcgacgccgaaagatggtccgagtgccggtattgctatgtgcaccgcgctggtttcttgcctgaccggtaacccggttcgtgccgatgtggcaatgaccggtgagatcactctgcgtggtcaggtactgccgatcggtggtttgaaagaaaaactcctggcagcgcatcgcggcgggattaaaacagtgctaattccgttcgaaaataaacgcgatctggaagagattcctgacaacgtaattgccgatctggacattcatcctgtgaagcgcattgaggaagttctgactctggcgctgcaaaatgaaccgtctggtatgcaggttgtgactgcaaaatag

SEQ ID NO: 16 - DNA, 954 bp, Escherichia coli
  source 1..954: /mol_type=“DNA” /organism=“Escherichia coli”
atgcgggcgaaacttctgggaatagtcctgacaacccctattgcgatcagctcttttgcttctaccgagactttatcgtttactcctgacaacataaatgcggacattagtcttggaactctgagcggaaaaacaaaagagcgtgtttatctagccgaagaaggaggccgaaaagtcagtcaactcgactggaaattcaataacgctgcaattattaaaggtgcaattaattgggatttgatgccccagatatctatcggggctgctggctggacaactctcggcagccgaggtggcaatatggtcgatcaggactggatggattccagtaaccccggaacctggacggatgaaagtagacaccctgatacacaactcaattatgccaacgaatttgatctgaatatcaaaggctggctcctcaacgaacccaattaccgcctgggactcatggccggatatcaggaaagccgttatagctttacagccagaggtggttcctatatctacagttctgaggagggattcagagatgatatcggctccttcccgaatggagaaagagcaatcggctacaaacaacgttttaaaatgccctacattggcttgactggaagttatcgttatgaagattttgaactcggtggcacatttaaatacagcggctgggtggaatcatctgataacgatgaacactatgacccgggaaaaagaatcacttatcgcagtaaggtcaaagaccaaaattactattctgttgcagtcaatgcaggttattacgtcacacctaacgcaaaagtttatgttgaaggcgcatggaatcgggttacgaataaaaaaggtaatacttcactttatgatcacaataataacacttcagactacagcaaaaatggagcaggtatagaaaactataacttcatcactactgctggtcttaagtacacattttaa

SEQ ID NO: 17 - DNA, 624 bp, Escherichia coli
  source 1..624: /mol_type=“DNA” /organism=“Escherichia coli”
atgtcaacgattattatggatttatgtagttacacccgactaggtttaaccgggtatctgttgagtagaggggttaaaaaaagagaaatcaacgacattgaaaccgttgatgaccttgccatagcttgtgattcacagcgcccttcagtggtgtttattaatgaggactgtttcatccacgatgcttctaacagtcagcgtatcaagctcatcattaatcaacatcccaatacgttatttatcgtttttatggcaattgccaatgttcattttgatgaatatctattggtcagaaaaaatttattgatcagttctaaatcgattaaaccggaatctctcgacgatatccttggcgatattctgaaaaaagagacaacgataacctcgtttttaaatatgccgacgttatcattgagccgaaccgaatcgagtatgttgcgaatgtggatggcaggtcagggaaccattcaaatctctgaccaaatgaatatcaaagccaagaccgtttcatcgcataaaggtaatattaaacgtaagatcaaaacgcataataaacaggttatctaccatgtcgtccgactgacggataatgtgactaatggtatttttgtcaacatgcgctaa

SEQ ID NO: 18 - DNA, 3567 bp, Escherichia coli
  source 1..3567: /mol_type=“DNA” /organism=“Escherichia coli”
atgttatgggccttaaatatttggacaggcccgcacagcaatggattaataacaatgatgaataaatccaattttgaattcctgaagggcgtcaacgacttcacttatgccatcgcctgtgcggcggaaaataactacccggatgatcccaacacgacgctgattaaaatgcgtatgtttggcgaagccacagcgaaacatcttggtctgttactcaacatccccccttgtgagaatcaacacgatctcctgcgtgaactcggcaaaatcgcctttgttgatgacaacatcctctctgtatttcacaaattacgccgcattggtaaccaggcggtgcacgaatatcataacgatctcaacgatgcccagatgtgcctgcgactcgggttccgcctggctgtctggtactaccgtctggtcactaaagattatgacttcccggtgccggtgtttgtgttgccggaacgtggtgaaaacctctatcaccaggaagtgctgacgctaaaacaacagcttgaacagcaggtgcgagaaaaagcgcagactcaggcagaagtcgaagcgcaacagcagaagctggttgccctgaacggctatatcgccattctggaaggcaaacagcaggaaaccgaagcgcaaacccaggctcgccttgcggcactggaagcacagctcgccgagaagaacgcggaactggcaaaacagaccgaacaggaacgtaaggcttaccacaaagaaattaccgatcaggccatcaagcgcacactcaaccttagcgaagaagagagtcgcttcctgattgatgcgcaactgcgtaaagcaggctggcaggccgacagcaaaaccctgcgcttctccaaaggcgcacgtccggaacccggcgtcaataaagccattgccgaatggccgaccggaaaagatgaaacgggtaatcagggctttgcggattatgtgctgtttgtcggcctcaaacccatcgcggtggtagaggcgaaacgtaacaatatcgacgttcccgccaggctcaatgagtcgtatcgctacagtaaatgtttcgataatggcttcctgcgggaaaccttgcttgagcactactcaccggatgaagtgcatgaagcagtgccagagtatgaaaccagctggcaggacaccagcggcaaacaacggtttaaaatccccttctgctactcgaccaacgggcgcgaataccgcgcaacaatgaagaccaaaagcggcatctggtatcgcgacgtgcgtgatacccgcaatatgtcgaaagccttacccgagtggcaccgcccggaagagctgctggaaatgctcggcagcgaaccgcaaaaacagaatcagtggtttgccgataaccctggcatgagcgagctgggcctgcgttattatcaggaagatgccgtccgcgcggttgaaaaggcaatcgtcaaggggcaacaagagatcctgctggcgatggcgaccggtaccggtaaaacccgtacggcaatcgccatgatgttccgcctgatccagtcccagcgttttaaacgcattctcttccttgtcgaccgccgttctcttggcgaacaggcgctgggcgcgtttgaagatacgcgtattaacggcgacaccttcaacagcattttcgacattaaagggctgacggataaattcccggaagacagcaccaaaattcacgttgccaccgtacagtcgctggtgaaacgcaccctgcaatcagatgaaccgatgccggtggcccgttacgactgtatcgtcgttgacgaagcgcatcgcggctatattctcgataaagagcagaccgaaggcgaactgcagttccgcagccagctggattacgtctctgcctaccgtcgcattctcgatcacttcgatgcggtaaaaatcgctctcaccgccaccccggcgctacatactgtgcagattttcggcgagccggtttaccgttatacctaccgtaccgcggttatcgacggttttctgatcgaccaggatccgcctattcagatcatcacccgcaacgcgcaggagggggtttatctctccaaaggcgagcaggtagagcgcatcagcccgcagggagaagtgatcaatgacaccctggaagacgatcaggattttgaagtcgccgactttaaccgtggcctggtgatcccggcgtttaaccgcgccgtctgtaacgaactcaccaattatcttgacccgaccggatcgcaaaaaacgctggtcttctgcgtcaccaatgcccatgccgatatggtggtggaagagctgcgtgccgcgttcaagaaaaagtatccgcaactggagcacgacgcgatcatcaagatcaccggtgatgccgataaagacgcgcgcaaagtgcagaccatgatcacccgcttcaataaagagcggctgcccaatatcgtggtaaccgtcgacctgctgacgaccggcgtcgatattccgtcgatctgtaatatcgtgttcctgcgtaaagtacgcagccgcattctgtacgaacagatgaaaggccgcgccacgcgcttatgcccggaggtgaataaaaccagctttaagatttttgactgtgtcgatatctacagcacgctggagagcgtcgacaccatgcgtccggtggtggtgcgcccgaaggtggaactgcaaacgctggtcaatgaaattaccgattcagaaacctataaaatcaccgaagcggatggccgcagttttgccgagcacagccatgaacaactggtggcgaagctccagcgtatcatcggtctggccacgtttaaccgtgaccgcagcgaaacgatagataaacaggtgcgtcgtctggatgagctatgccaggacgcggcgggcgtgaactttaacggcttcgcctcgcgcctgcgggaaaaagggccgcactggagcgccgaagtctttaacaaactgcctggctttatcgcccgtctggaaaagctgaaaacggacatcaacaacctgaatgatgcgccgatcttcctcgatatcgacgatgaagtggtgagtgtaaaatcgctgtacggtgattacgacacgccgcaggatttcctcgaagcctttgactcgctggtgcaacgttccccgaacgcgcaaccggcattgcaggcagttattaatcgcccgcgcgatctcacccgtaaagggctggtcgagctacaggagtggtttgaccgccagcactttgaggaatcttccctgcgcaaagcatggaaagagacgcgcaatgaagatatcgccgcccggctgattggtcatattcgccgcgctgcggtgggcgatgcgctgaaaccgtttgaggaacgtgtcgatcacgcgctgacgcgcattaagggcgaaaacgactggagcagcgagcaattaagctggctcgatcgtttagcgcaggcgctgaaagagaaagtggtgctcgacgacgatgtcttcaaaaccggcaacttccaccgtcgcggcgggaaggcgatgctgcaaagaacctttgacgataatctcgataccctgctgggcaaattcagcgattatatctgggacgagctggcctga

SEQ ID NO: 19 - DNA, 915 bp, Escherichia coli
  source 1..915: /mol_type=“DNA” /organism=“Escherichia coli”
atgacggttcctacctatgacaaatttattgaacctgttctgcgttatctggcaacaaaaccggaaggtgcagccgcgcgtgatgttcatgaggctgccgcggatgcattaggactggatgacagccagcgagcgaaagtcattaccagcggacaacttgtttataaaaatcgtgcaggctgggcgcatgaccgtttaaaacgtgccgggttgtcgcaaagtttgtcgcgtggcaaatggtgcctgactcctgcgggttttgactgggttgcgtctcatccccagccaatgacggagcaggagacgaaccatctggccttcgcttttgtgaatgtcaaacttaagtcacggccggatgccgtcgatttagatccgaaagccgactctcccgatcatgaagaacttgcaaagagcagcccggacgatcggttagatcaggcgctaaaagagcttcgtgatgcggtggctgatgaggttctggaaaacttattgcaggtttctccttcgcgctttgaagtcattgttctggatgttttgcatcgcctggggtatggcggccaccgtgatgatttgcagcgtgttggcggtactggagatggtggcatcgatggtgtgatatcgcttgataaacttggcctggagaaagtttatgttcaggcaaaacgttggcagaatactgtaggcaggccagaattacaggcattttacggcgcactggctgggcaaaaagcgaaacgtggggtgtttattaccacttctggatttacttctcaggcgcgtgactttgcccaatccgtcgagggtatggtgttggttgatggggaacgcctggtgcacttaatgatcgaaaacgaagtaggggtttcttcacgtttgttgaaggtgccgaaactggatatggactattttgagtga

SEQ ID NO: 20 - DNA, 708 bp, Escherichia coli
  source 1..708: /mol_type=“DNA” /organism=“Escherichia coli”
atgtaccgttatttgtctattgctgcggtggtactgagcgcagcattttccggcccggcgttggccgaaggtatcaatagtttttctcaggcgaaagccgcggcggtaaaagtccacgctgacgcgcccggtacgttttattgcggatgtaaaattaactggcagggcaaaaaaggcgttgttgatctgcaatcgtgcggctatcaggtgcgcaaaaatgaaaaccgcgccagccgcgtagagtgggaacatgtcgttcccgcctggcagttcggtcaccagcgccagtgctggcaggacggtggacgtaaaaactgcgctaaagatccggtctatcgcaagatggaaagcgatatgcataacctgcagccgtcagtcggtgaggtgaatggcgatcgcggcaactttatgtacagccagtggaatggcggtgaaggccagtacggtcaatgcgccatgaaggtcgatttcaaagaaaaagctgccgaaccaccagcgcgtgcacgcggtgccattgcgcgcacctacttctatatgcgcgaccaatacaacctgacactctctcgccagcaaacgcagctgttcaacgcatggaacaagatgtatccggttaccgactgggagtgcgagcgcgatgaacgcatcgcgaaggtgcagggcaatcataacccgtatgtgcaacgcgcttgccaggcgcgaaagagctaa

SEQ ID NO: 21 - DNA, 1062 bp, Escherichia coli
  source 1..1062: /mol_type=“DNA” /organism=“Escherichia coli”
atggctatcgacgaaaacaaacagaaagcgttggcggcagcactgggccagattgagaaacaatttggtaaaggctccatcatgcgcctgggtgaagaccgttccatggatgtggaaaccatctctaccggttcgctttcactggatatcgcgcttggggcaggtggtctgccgatgggccgtatcgtcgaaatctacggaccggaatcttccggtaaaaccacgctgacgctgcaggtgatcgccgcagcgcagcgtgaaggtaaaacctgtgcgtttatcgatgctgaacacgcgctggacccaatctacgcacgtaaactgggcgtcgatatcgacaacctgctgtgctcccagccggacaccggcgagcaggcactggaaatctgtgacgccctggcgcgttctggcgcagtagacgttatcgtcgttgactccgtggcggcactgacgccgaaagcggaaatcgaaggcgaaatcggcgactctcacatgggccttgcggcacgtatgatgagccaggcgatgcgtaagctggcgggtaacctgaagcagtccaacacgctgctgatcttcatcaaccagatccgtatgaaaattggtgtgatgttcggtaacccggaaaccactaccggtggtaacgcgctgaaattctacgcctctgttcgtctcgacatccgtcgtatcggcgcggtgaaagagggcgaaaacgtggtgggtagcgaaacccgcgtgaaagtggtgaagaacaaaatcgctgcgccgtttaaacaggctgaattccagatcctctacggcgaaggtatcaacttctacggcgaactggttgacctgggcgtaaaagagaagctgatcgagaaagcaggcgcgtggtacagctacaaaggtgagaagatcggtcagggtaaagcgaatgcgactgcctggctgaaagataacccggaaaccgcgaaagagatcgagaagaaagtacgtgagttgctgctgagcaacccgaactcaacgccggatttctctgtagatgatagcgaaggcgtagcagaaactaacgaagatttttaa

SEQ ID NO: 22 - DNA, 306 bp, Escherichia coli
  source 1..306: /mol_type=“DNA” /organism=“Escherichia coli”
atgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataa

SEQ ID NO: 23 - DNA, 219 bp, Escherichia coli
  source 1..219: /mol_type=“DNA” /organism=“Escherichia coli”
atgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtga

SEQ ID NO: 24 - DNA, 59 bp, artificial sequences
  source 1..59: /mol_type=“DNA” /note=“primers XisgalKstart” /organism=“artificial sequences”
gggggtaaatcccggcgctcatgacttcgccttcttcccagaattcctgttgacaatta

SEQ ID NO: 25 - DNA, 60 bp, artificial sequences
  source 1..60: /mol_type=“DNA” /note=“XisgalK stop” /organism=“artificial sequences”
gttctgattattggaaatcttctttgccctccagtgtgagcagcactgtcctgctccttg

SEQ ID NO: 26 - DNA, 20 bp, artificial sequences
  source 1..20: /mol_type=“DNA” /note=“Xis1 primer” /organism=“artificial sequences”
gtcttcaagtggagcatcag

SEQ ID NO: 27 - DNA, 20 bp, artificial sequences
  source 1..20: /mol_type=“DNA” /note=“Xis4 primer” /organism=“artificial sequences”
accaggactatccgtatgac

SEQ ID NO: 28 - DNA, 35 bp, artificial sequences
  source 1..35: /mol_type=“DNA” /note=“Xis2 primer” /organism=“artificial sequences”
ccaaacggaacagatgaagaaggcgaagtcatgag

SEQ ID NO: 29 - DNA, 35 bp, artificial sequences
  source 1..35: /mol_type=“DNA” /note=“Xis3 primer” /organism=“artificial sequences”
gacttcgccttcttcatctgttccgtttggcttcc

SEQ ID NO: 30 - DNA, 21 bp, artificial sequences
  source 1..21: /mol_type=“DNA” /note=“Xis6 primer” /organism=“artificial sequences”
gtaatggaaagctggtagtcg

SEQ ID NO: 31 - DNA, 20 bp, artificial sequences
  source 1..20: /mol_type=“DNA” /note=“Xis5 primer” /organism=“artificial sequences”
cagccgtaagtcttgatctc

SEQ ID NO: 32 - DNA, 20 bp, artificial sequences
  source 1..20: /mol_type=“DNA” /note=“Xis7 primer” /organism=“artificial sequences”
cagcaggcatgatccaagag

SEQ ID NO: 33 - DNA, 35 bp, artificial sequences
  source 1..35: /mol_type=“DNA” /note=“Xis2b primer” /organism=“artificial sequences”
tttgccctccagtgtgaagaaggcgaagtcatgag

SEQ ID NO: 34 - DNA, 39 bp, artificial sequences
  source 1..39: /mol_type=“DNA” /note=“Xis8 primer” /organism=“artificial sequences”
ctcatgacttcgccttcttcacactggagggcaaagaag

SEQ ID NO: 35 - DNA, 624 bp, Enterobacteria phage lambda
  source 1..624: /mol_type=“DNA” /organism=“Enterobacteria phage lambda”
atgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttag

SEQ ID NO: 36 - DNA, 144 bp, Enterobacteria phage lambda
  source 1..144: /mol_type=“DNA” /organism=“Enterobacteria phage lambda”
atggatcaaacacttatggctatccagactaaattcactatcgccacttttattggcgatgaaaagatgtttcgtgaagccgtcgacgcttataaaaaatggatattaatactgaaactgagatcaagcaaaagcattcactaa